(19) United States
(12) Patent Application Publication
(10) Pub. No.: US 2008/0125782 A1
(43) Pub. Date: May 29, 2008

(54) METHOD AND APPARATUS FOR REMOVING AN EXTENSION FROM A PROSTHESIS

(75) Inventors: Mark A. RYDELL, Golden Valley, MN (US); Khin MYINT, Shakopee, MN (US); Robert Garryl HUDGINS, Burnsville, MN (US); Erik O. MARTZ, Savage, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: DISC DYNAMICS, INC., Eden Prairie, MN (US)

(21) Appl. No.: 11/564,708

(22) Filed: Nov. 29, 2006

(51) Int. Cl. A61B 17/56

(52) U.S. Cl. 606/79

(57) ABSTRACT

An instrument for cutting an extension, such as a catheter or biomaterial, coupled to a prosthesis, such as a prosthesis located in an intervertebral disc space, that includes at least one axial member having a distal opening and at least one cutting device, such as for example a wire or blade, coupled to the at least one axial member that is capable of moving across at least a portion of the distal opening when a force is applied to the at least one cutting wire. A method for cutting a catheter is also disclosed. The removal instrument may be inserted into an introduction lumen. A force is applied to the at least one cutting device after the catheter is in the distal opening.

### FIELD OF THE INVENTION

The present invention relates to a method and apparatus for removing an extension or portion thereof, such as a catheter or biomaterial, from a prosthesis, such as an intervertebral prosthesis.

### BACKGROUND OF THE INVENTION

The intervertebral discs, which are located between adjacent vertebrae in the spine, provide structural support for the spine as well as the distribution of forces exerted on the spinal column. Intervertebral discs are, however, susceptible to a number of injuries. Disc herniation occurs when the nucleus begins to extrude through an opening in the annulus, often to the extent that the herniated material impinges on nerve roots in the spine or spinal cord. The posterior and posterolateral portions of the annulus are most susceptible to attenuation or herniation, and therefore, are more vulnerable to hydrostatic pressures exerted by vertical compressive forces on the intervertebral disc. Various injuries and deterioration of the intervertebral disc and annulus fibrosus are discussed by Osti et al., Annular Tears and Disc Degeneration in the Lumbar Spine, J. Bone and Joint Surgery, 74-B(5), (1982) pp. 678-682; Osti et al., Annulus Tears and Intervertebral Disc Degeneration, Spine, 15(8) (1990) pp. 762-767; Kamblin et al., Development of Degenerative Spondylosis of the Lumbar Spine after Partial Discectomy, Spine, 20(5) (1995) pp. 599-607.

Many treatments for intervertebral disc injury have involved the use of nucleus prostheses or disc spacers that are inserted into the nuclear space of the intervertebral disc. A variety of prosthetic nuclear implants are known in the art. Sometimes these prosthetic nuclear implants are inserted into the intervertebral disc space as an empty vessel, similar to a deflated balloon, which is attached to a catheter. Sometimes, the prosthetic nuclear implants are introduced into the intervertebral space with an introduction lumen, particularly if the procedure is performed using a minimally invasive technique. Once the empty vessel is inserted into the nuclear space, a material is injected through the catheter into the empty vessel to fill the vessel. The material may be cured, for example, in order to form a finished nucleus prosthesis. For example, U.S. Pat. No. 5,047,055 (Bao et al.) teaches filling the nucleus prosthesis with a swellable hydrogel.

The catheter must then be removed from the nucleus prosthesis before the surgery is complete. This has been accomplished in the past by inserting into the introduction lumen a device that has a tubular member with a flat blade at the distal end of the tubular member. Once the blade reaches the proximal end of the catheter, the surgeon rotates the device to spiral cut through the catheter until it has reached the nucleus prosthesis and has left very little catheter on the nucleus prosthesis.

### SUMMARY OF THE INVENTION

The present invention is directed to a method and apparatus for removing an extension or portion thereof, such as a catheter or biomaterial, from a prosthesis, such as an intervertebral prosthesis. The present method and apparatus are particularly well suited to cut the delivery catheter on spinal implants that are filled in situ with a curable biomaterials, such as for example, the prostheses disclosed in U.S. Pat. Nos. 5,556,429 (Felt); 6,306,177 (Felt, et al.); 6,248,131 (Felt, et al.); 5,795,353 (Felt); 6,079,868 (Rydell); 6,443,988 (Felt, et al.); 6,140,452 (Felt, et al.); 5,888,220 (Felt, et al.); 6,224,630 (Bao, et al.); 7,001,431 (Felt et al.); U.S. patent application Ser. No. 11/268,786 entitled MULTI-LUMEN MOLD FOR INTERVERTEBRAL PROSTHESIS AND METHOD OF USING SAME filed Nov. 8, 2005; U.S. patent application Ser. No. 11/304,053 entitled TOTAL NUCLEUS REPLACEMENT (TNR) METHOD filed on Dec. 16, 2004; U.S. patent application Ser. No. 10/984,493 entitled MULTI-STAGE BIOMATERIAL INJECTOR SYSTEM FOR SPINAL IMPLANTS filed on Nov. 9, 2004; and U.S. patent application Ser. No. 10/984,566 entitled MULTI-STAGE BIOMATERIAL INJECTOR SYSTEM FOR SPINAL IMPLANTS filed on Nov. 9, 2004, U.S. patent application Ser. No. 11/277,887 entitled INTERVERTEBRAL DISC PROSTHESIS, filed on Mar. 29, 2006, and U.S. patent application Ser. No. 11/420,055 entitled MOLD ASSEMBLY FOR INTERVERTEBRAL PROSTHESIS, filed on May 24, 2006, all of which are hereby incorporated by reference.

In one embodiment, the present invention is directed toward an instrument for cutting an extension, such as a catheter or biomaterial, coupled to a prosthesis, such as a prosthesis located in an intervertebral disc space. This embodiment includes at least one axial member having a proximal end, a distal end, a distal opening at the distal end, and an inside diameter adapted to receive the extension; and at least one cutter positioned to traverse at least a portion of the distal opening when a force is applied near the proximal end of the axial member. This embodiment may be particularly useful in procedures using minimally invasive techniques.

In one embodiment, the axial member may include a tubular member. In another embodiment, the axial member may include a rod member and an extension engaging member at the distal end of the rod member. The extension engaging member may include an eyelet, or a hook, such as a circular, square-shaped or other appropriately shaped hook.

In one embodiment, the force may be torque. The torque may be applied using a mechanized device, such as a drill.

In one embodiment, the tubular member may have a diameter of less than about 10 millimeters.

In one embodiment, the cutter may be a cutting wire. The cutting wire may include one or more cutting loops that are capable of moving across the distal opening when a tension force is applied to the cutting wire.

In one embodiment, the tubular member optionally includes at least one wire fastening device located on the distal perimeter edge. The cutting wire is capable of moving through the at least one wire fastening device relative to the at least one tubular member.

In another embodiment, the instrument includes an outer tubular member and an inner tubular member inside the outer tubular member. One or more cutting wires are coupled to both the outer tubular member and to the inner tubular member. Movement of the inner tubular member relative to the outer tubular member actuates the cutting action.

The present invention is also directed to an instrument for cutting a catheter leading to a nucleus prosthesis that includes at least one tubular member having a distal opening and at least one blade coupled to the at least one tubular member that is capable of moving across at least a portion of the distal opening when a force is applied.

In another embodiment, the instrument includes an outer tubular member and an inner tubular member inside of the outer tubular member. The outer tubular member may include an interior surface that tapers toward a central axis of the outer tubular member. The blade may be coupled to the inner tubular member or the outer tubular member. In this embodiment, the inner tubular member may include two, four, eight or more than one blade. The blades may be a variety of shapes, such as for example parabolic, triangular, flat, serrated, or another suitable shape.

In another embodiment, the tubular member may include at least one housing and the at least one blade may include at least one blade handle such that when the force is applied to the at least one blade handle, the at least one blade moves along the at least one housing to the distal opening. The at least one blade may include a first blade and a second blade that may or may not overlap across the distal opening.

In yet another embodiment utilizing an outer tubular member and an inner tubular member inside of the outer tubular member, the at least one blade may include a tapering surface that is pivotally coupled to a perimeter of the inner tubular member. The outer tubular member may include a wedge having an inclined surface that is capable of engaging with the tapering surface to push the at least one blade across at least a portion of the distal opening.

In another embodiment, at least one blade is formed on a distal end of the at least one tubular member and the instrument further includes at least one removable blade guard located inside the at least one tubular member that covers the at least one blade.

In still another embodiment, the invention is directed to an instrument for cutting an extension leading to a prosthesis that includes at least one tubular member having a perimeter at a distal end and at least one transverse cutting device attached to the perimeter which forms a first distal opening and a second distal opening. The cutter may be a blade or a cutting wire.

In another embodiment, the invention is directed to an instrument for cutting an extension coupled to a prosthesis, that includes an inner axial member having a proximal end and a distal end, an outer tubular member having an inside surface adapted to receive the inner axial member, a proximal end, and a distal end, and a cutter positioned at the distal end of the inner axial member or the outer tubular member and adapted to engage with the extension when a force is applied near the proximal end of the inner axial member or the outer tubular member.

In one embodiment, the inner axial member may further include a distal opening at the distal end, a lip adjacent to the distal opening, and an inside diameter adapted to receive the extension and wherein the cutter is positioned on the lip, such that when the force is applied near the proximal end of the outer tubular member, the cutter engages with the extension.

In one embodiment, the plane of the distal opening is perpendicular to the central axis of the axial member. In another embodiment, the distal opening is angled relative to the central axis of the axial member.

In another embodiment, the outer tubular member may include a perimeter having an inner edge that is capable of receiving the extension through a distal opening at the distal end and where the cutter is positioned on the inner edge such that when the force is applied near the proximal end of the inner axial member, the cutter engages with the extension. In still another embodiment, outer tubular member may further include a perimeter having an inner edge that is capable of receiving the extension through the distal opening and where the cutter is positioned on the distal end of the inner axial member such that when the force is applied near the proximal end of the inner axial member, the cutter engages with the extension.

In yet another embodiment, the invention is directed to a method of cutting a catheter, the catheter coupled to a prosthesis located in an intervertebral disc space, the method including the steps of positioning at least one tubular member around the catheter so that a distal opening of the tubular member is near the prosthesis, and applying a force near the proximal end of the tubular member so that at least one cutter traverses at least a portion of the distal opening such that at least a portion of the catheter is cut. The at least one cutter may be a cutting wire or a blade. The method may also include the step of heating, vibrating, oscillating, applying rotation or translation (such as wiping or pinching), ultrasonic waves, or radiofrequency energy to the cutter before or while the force or torque is applied.

As used herein the following words and terms shall have the meanings ascribed below:

“biomaterial” will generally refer to a material that is capable of being introduced to the site of a joint and cured to provide desired physical-chemical properties in vivo. In one embodiment the term will refer to a material that is capable of being introduced to a site within the body using minimally invasive mechanism, and cured or otherwise modified in order to cause it to be retained in a desired position and configuration. Generally such biomaterials are flowable in their uncured form, meaning they are of sufficient viscosity to allow their delivery through a delivery tube of on the order of about 1 mm to about 6 mm inner diameter, and preferably of about 2 mm to about 3 mm inner diameter. Such biomaterials are also curable, meaning that they can be cured or otherwise modified, in situ, at the tissue site, in order to undergo a phase or chemical change sufficient to retain a desired position and configuration;

“cut” is used herein to deforming the extension such that it can be more easily removed from a prosthesis. In one embodiment, cutting may include slicing an extension or a portion thereof. In other embodiments, cutting may include smearing, separating, or pinching-off an extension or a portion thereof. These other embodiments may be particularly useful if the extension is formed of cured or partially-cured biomaterial.

“cure” and inflections thereof, will generally refer to any chemical transformation (e.g., reacting or cross-linking), physical transformation (e.g., hardening or setting), and/or mechanical transformation (e.g., drying or evaporating) that allows the biomaterial to change or progress from a first physical state or form (generally liquid or flowable) that allows it to be delivered to the site, into a more permanent second physical state or form (generally solid or gelled) for final use in vivo. When used with regard to the method of the invention, for instance, “curable” can refer to uncured biomaterial, having the potential to be cured in vivo (as by catalysis or the application of a suitable energy source), as well as to the biomaterial in the process of curing. As further described herein, in selected embodiments the cure of a biomaterial can generally be considered to include three stages, including (a) the onset of gelation, (b) a period in which gelation occurs and the biomaterial becomes sufficiently tack-free to permit shaping or cutting, and (c) complete cure to the point where the biomaterial has been finally shaped for its intended use.

“extension” refers to a portion of a catheter or biomaterial extending from a prosthesis, such as an intervertebral prosthesis, or cured or partially-cured biomaterial extending from a prosthesis.

“minimally invasive mechanism” refers to a surgical mechanism, such as microsurgical, percutaneous, or endoscopic or arthroscopic surgical mechanism, that can be accomplished with minimal disruption to the annular wall (e.g., incisions of less than about 4 cm and preferably less than about 2 cm). In some embodiments, minimally invasive mechanisms also refers to minimal disruption of the pertinent musculature, for instance, without the need for open access to the tissue injury site or through minimal skin incisions. Such surgical mechanism are typically accomplished by the use of visualization such as fiberoptic or microscopic visualization, and provide a post-operative recovery time that is substantially less than the recovery time that accompanies the corresponding open surgical approach.

“mold” will generally refer to the portion or portions of an apparatus of the invention used to receive, constrain, shape and/or retain a flowable biomaterial in the course of delivering and curing the biomaterial in situ. A mold may include or rely upon natural tissues (such as the annular shell of an intervertebral disc) for at least a portion of its structure, conformation or function. The mold, in turn, is responsible, at least in part, for determining the position and final dimensions of the cured prosthetic implant. As such, its dimensions and other physical characteristics can be predetermined to provide an optimal combination of such properties as the ability to be delivered to a site using minimally invasive mechanisms, filled with biomaterial, prevent moisture contact, and optionally, then remain in place as or at the interface between cured biomaterial and natural tissue. In one embodiment the mold material can itself become integral to the body of the cured biomaterial. The mold can be elastic or inelastic, permanent or bio-reabsorbable, porous or non-porous.

“traverse” will generally refer to moving into or across the cross-sectional space of the distal opening or the extension, whether in front, behind, or at the perimeter of the tubular member.

### BRIEF DESCRIPTION OF THE DRAWINGS

FIG. 1 illustrates a side sectional view of an intervertebral space containing a prosthesis.

FIG. 2A illustrates a perspective view of one embodiment of a removal instrument that includes a cutting wire in accordance with the present invention.

FIG. 2B illustrates a perspective view of another embodiment of a removal instrument that includes a cutting wire in accordance with the present invention.

FIG. 2C illustrates a perspective view of still another embodiment of a removal instrument that includes a cutting wire in accordance with the present invention.

FIG. 2D illustrates a perspective view of another embodiment of a removal instrument that includes a cutting wire in accordance with the present invention.

FIG. 2E illustrates a perspective view of still another embodiment of a removal instrument that includes a cutting wire in accordance with the present invention.

FIG. 2F illustrates a cross sectional view of the removal instrument of FIG. 2E.

FIG. 2G illustrates a perspective view of yet another embodiment of a removal instrument that includes a cutting wire in accordance with the present invention.

FIG. 2H illustrates an end view of another embodiment of a removal instrument that includes a cutting wire in accordance with the present invention.

FIG. 3 illustrates a perspective view of yet another embodiment of a removal instrument that includes a cutting wire in accordance with the present invention.

FIG. 4 illustrates a perspective view of another embodiment of a removal instrument that includes a cutting wire in accordance with the present invention.

FIG. 5 illustrates a side view of still another embodiment of a removal instrument that includes a cutting wire in accordance with the present invention.

FIG. 6 illustrates an enlarged side view of the distal end of the removal instrument of FIG. 5.

FIG. 7 illustrates a side view of the removal instrument of FIG. 5 advancing over a catheter toward a prosthesis.

FIG. 8 illustrates a side view of the removal instrument of FIG. 5 engaged with the prosthesis.

FIG. 9 illustrates a perspective view of yet another embodiment of a removal instrument that includes a cutting wire in accordance with the present invention.

FIG. 10 illustrates a side view of the removal instrument of FIG. 9 advancing over a catheter toward a prosthesis.

FIG. 11 illustrates a perspective view of the removal instrument of FIG. 9 after a force has been applied to the cutting wire.

FIG. 12 illustrates a perspective view of an embodiment of a removal instrument that includes two cutting wires in accordance with the present invention.

FIG. 13 illustrates an exploded view of another embodiment of a removal instrument that includes two cutting wires in accordance with the present invention.

FIG. 14 illustrates a perspective view of the removal instrument of FIG. 13.

FIG. 15 illustrates a perspective view of the removal instrument of FIG. 13 while a force is applied to the cutting wires.

FIG. 16 illustrates a side sectional view of one embodiment of a removal instrument that includes a plurality of blades, an inner tubular member, and an outer tubular member in accordance with the present invention.

FIG. 17 illustrates a side sectional view of the removal instrument of FIG. 16 once the blades have traversed the distal end of the outer tubular member.

FIG. 18 illustrates a perspective view of the removal instrument of FIG. 16 once the blades have traversed the distal end of the outer tubular member.

FIG. 19 illustrates a side view of one embodiment of the inner tubular member of the removal instrument of FIG. 16 containing 4 parabolic-shaped blades in accordance with the present invention.

FIG. 20 illustrates a side view of another embodiment of the inner tubular member of the removal instrument of FIG. 16 containing 4 triangular-shaped blades in accordance with the present invention.

FIG. 21 illustrates a side view of one embodiment of the inner tubular member of the removal instrument of FIG. 16 containing 4 flat-shaped blades in accordance with the present invention.

FIG. 22 illustrates a side view of another embodiment of a removal instrument that includes a plurality of blades, an inner tubular member, and an outer tubular member in accordance with the present invention.

FIG. 23 illustrates an enlarged view of the distal end of the removal catheter of FIG. 22.

FIG. 24 illustrates an end view of the distal end of the removal catheter of FIG. 22.

FIG. 25 illustrates an enlarged view of the distal end of the removal catheter of FIG. 22 after a force is applied to the outer tubular member.

FIG. 26 illustrates a side view of one embodiment of a removal instrument that includes two blades, an inner tubular member, and an outer tubular member before a force is applied to the outer tubular member, in accordance with the present invention.

FIG. 27 illustrates a side view of the removal instrument of FIG. 26 after a force is applied to the outer tubular member such that the blades cut the extension.

FIG. 28 illustrates a perspective view of one embodiment of a removal instrument that includes two blades, a tubular member, and housing in accordance with the present invention.

FIG. 29 illustrates a perspective view of another embodiment of a removal instrument that includes two blades, a tubular member, and housing in accordance with the present invention.

FIG. 30 illustrates an exploded view of still another embodiment of a removal instrument that includes two blades, an inner tubular member, and an outer tubular member before a force is applied to the outer tubular member, in accordance with the present invention.

FIG. 31 illustrates a perspective view of the removal instrument of FIG. 31 before a force is applied to the outer tubular member, in accordance with the present invention.

FIG. 32 illustrates a perspective view of the removal instrument of FIG. 31 after a force is applied to the outer tubular member, in accordance with the present invention.

FIG. 33 an exploded view of yet another embodiment of a removal instrument that includes two blades, an inner tubular member, and an outer tubular member before a force is applied to the outer tubular member, in accordance with the present invention.

FIG. 34 illustrates an enlarged perspective view of the removal instrument of FIG. 33 before a force is applied to the outer tubular member, in accordance with the present invention.

FIG. 35 illustrates a perspective view of the removal instrument of FIG. 33 before a force is applied to the outer tubular member, in accordance with the present invention.

FIG. 36 illustrates a perspective view of one embodiment of a removal instrument that includes a blade, an inner tubular member, and an outer tubular member having a wedge before a force is applied to the inner or outer tubular members, in accordance with the present invention.

FIG. 37 illustrates a perspective view of the removal instrument of FIG. 36 after a force has been applied to the inner or outer tubular members.

FIG. 38 illustrates a perspective view of another embodiment of a removal instrument that includes a blade and a blade guard in accordance with the present invention.

FIG. 39 illustrates a sectional view of the removal instrument of FIG. 38.

FIG. 40 illustrates a perspective view of one embodiment of a removal instrument that includes two blades in accordance with the present invention.

FIG. 41 illustrates a perspective view of one embodiment of a removal instrument that includes a cutting wire in accordance with the present invention.

FIG. 42A illustrates a side sectional view of one embodiment of an inner tubular member in accordance with the present invention for use in a removal instrument illustrated in FIG. 42B.

FIG. 42B illustrates a side sectional view of another embodiment of a removal instrument that includes an inner tubular member with a blade and an outer tubular member before a force is applied to the outer tubular member, in accordance with the present invention.

FIG. 42C illustrates an enlarged side sectional view of the removal instrument of FIG. 42B after a force has been applied to the outer tubular member.

FIG. 43A illustrates a perspective view of another embodiment of a removal instrument that includes a blade on an outer tubular member and an inner axial member, in accordance with the present invention.

FIG. 43B illustrates a side view of the removal instrument of FIG. 43A.

FIG. 43C illustrates another embodiment of an outer tubular member in accordance with the present invention for use with the removal instrument of FIG. 43A.

FIG. 43D illustrates still another embodiment of an outer tubular member in accordance with the present invention for use with the removal instrument of FIG. 43A.

FIG. 44 illustrates a perspective view of yet another embodiment of a removal instrument that includes an outer tubular member and an inner axial member with a blade, in accordance with the present invention.

FIG. 45A illustrates a side view of one embodiment of an inner axial member including a blade in accordance with the present invention.

FIG. 45B illustrates a sectional side view of the distal end of the inner axial member of FIG. 45A.

FIG. 45C illustrates a side view of one embodiment of an outer tubular member in accordance with the present invention.

FIG. 45D illustrates a side view of another embodiment of an inner axial member including a blade in accordance with the present invention.

FIG. 45E illustrates a perspective view of the inner axial member of FIG. 45D.

FIG. 45F illustrates a side view with partial sectional view of another embodiment of an outer tubular member in accordance with the present invention.

FIG. 46A illustrates an exploded view of one embodiment of a removal instrument that includes three cutting wires in accordance with the present invention.

FIG. 46B illustrates a perspective view of the removal instrument of FIG. 46 A as a force is applied to the cutting wires.

FIG. 47 illustrates a perspective view of yet another embodiment of a removal instrument including a cutting wire in accordance with the present invention.

FIG. 48 illustrates a perspective view of another embodiment of a removal instrument including a blade in accordance with the present invention.

### DETAILED DESCRIPTION

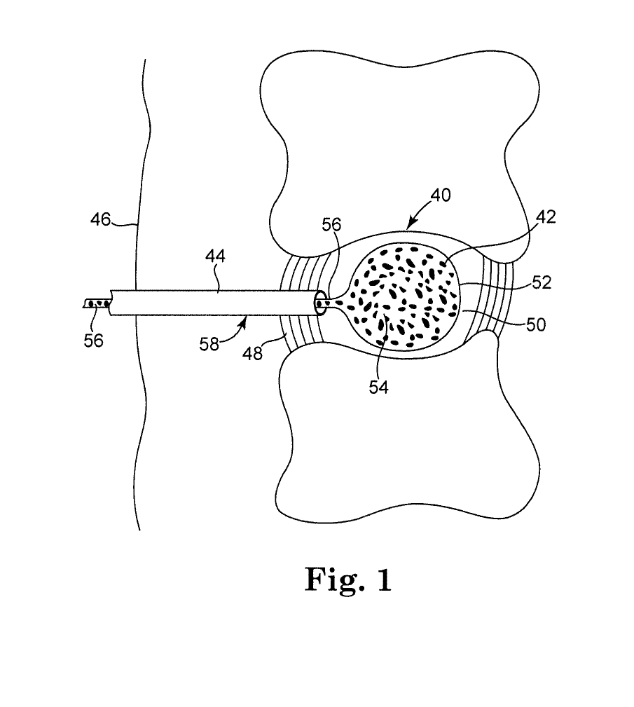

FIG. 1 is a side sectional view of an intervertebral space 40 containing an exemplary prosthesis 42. Lumen 44 extends from exterior of the patient 46 through the annulus 48 and into the nuclear cavity 50. In the illustrated embodiment, the exemplary prosthesis 42 is a mold 52 fluidly coupled to one or more delivery catheters 56. Curable biomaterial 54 is delivered to the mold 52 through the delivery catheter 56. Once the biomaterial 54 is at least partially cured, the delivery catheter 56 needs to be removed from the patient 46. The present invention is directed to various methods and devices for removing the delivery catheter 56 containing the at least partially cured biomaterial or at least partially cured biomaterial without a catheter. In the illustrated embodiment, the present removal instrument is either inserted into the lumen 44 with the delivery catheter 56 or the lumen 44 is part of the removal instrument. As shown in the illustrated embodiment, the lumen 44 extends into the nuclear cavity 50. However, in other embodiments, the lumen 44 may extend to a point outside of the nuclear cavity 50, such as to point 58, or to a location between point 58 and the nuclear cavity 50, such as the interior of the annulus 48.

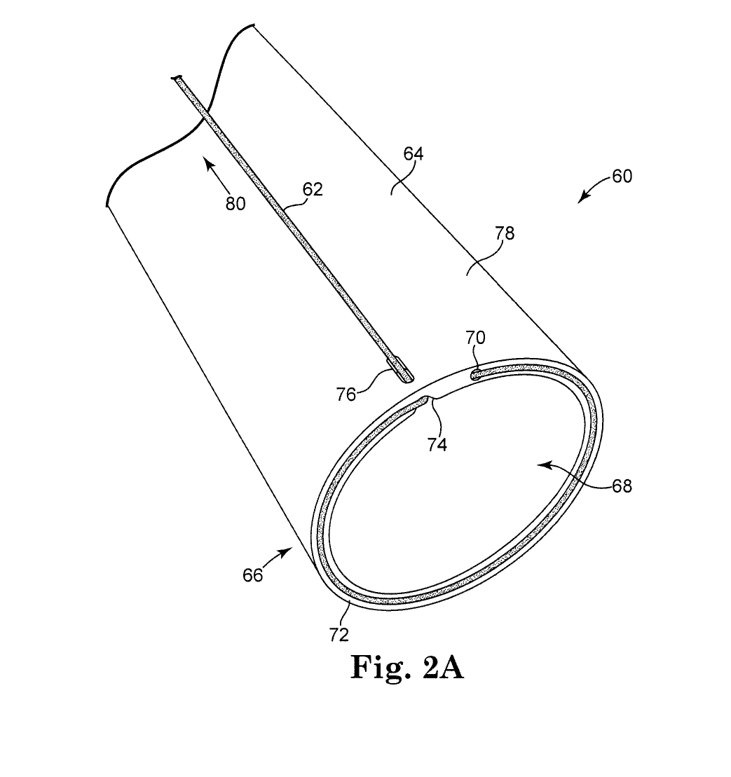

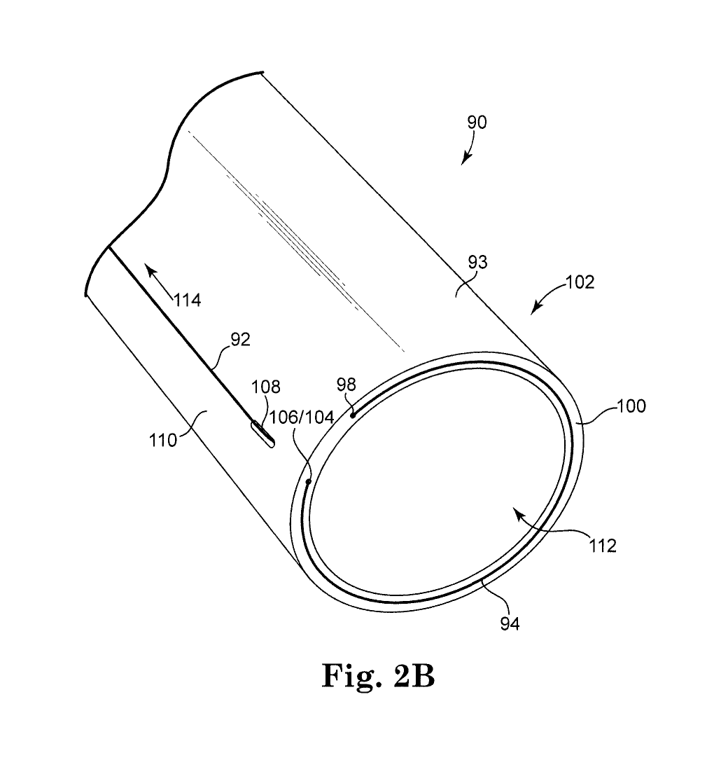

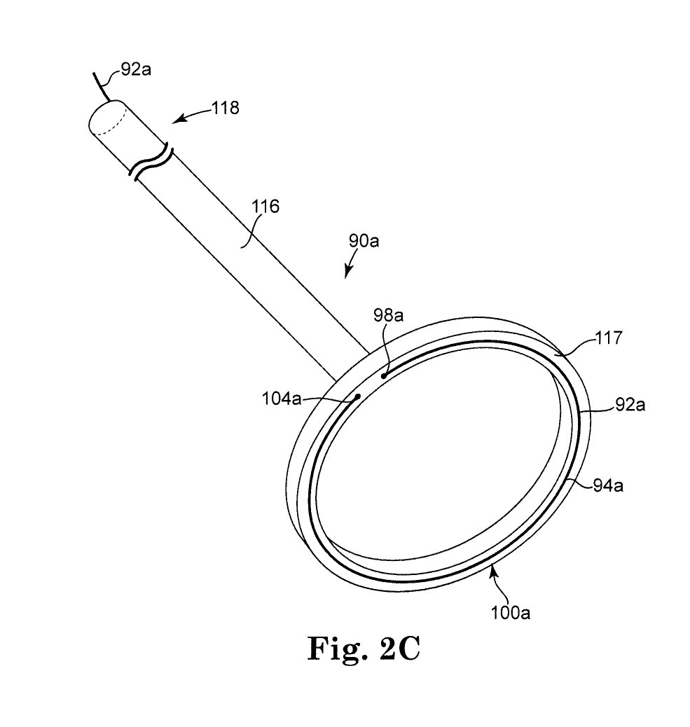

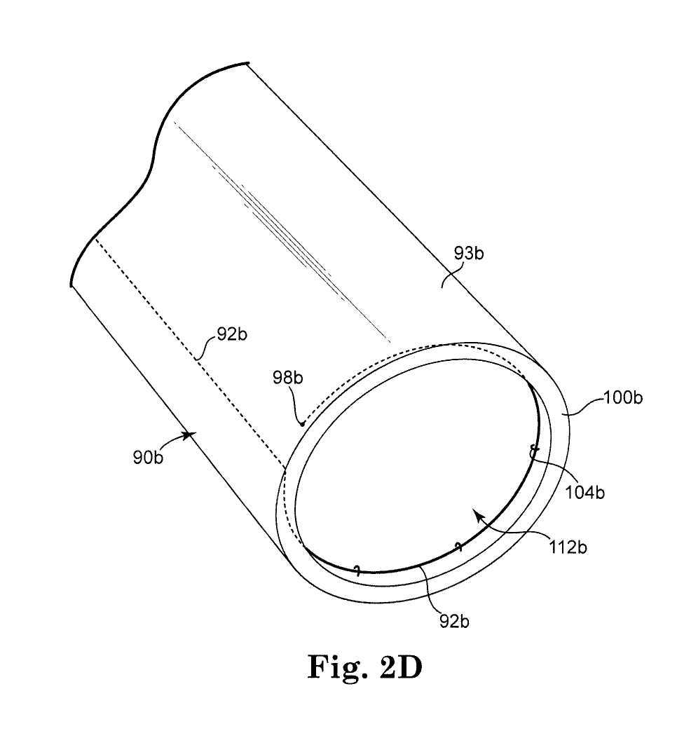

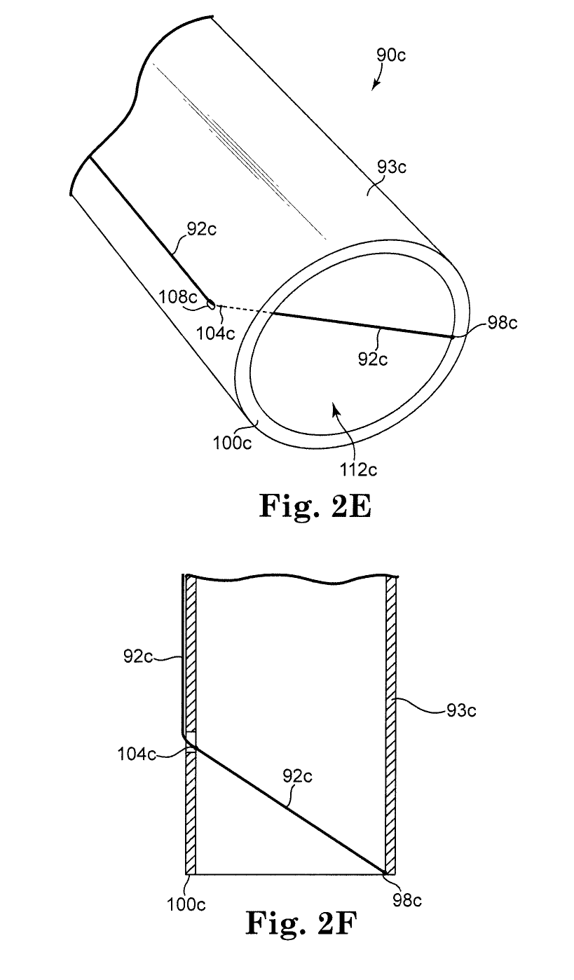

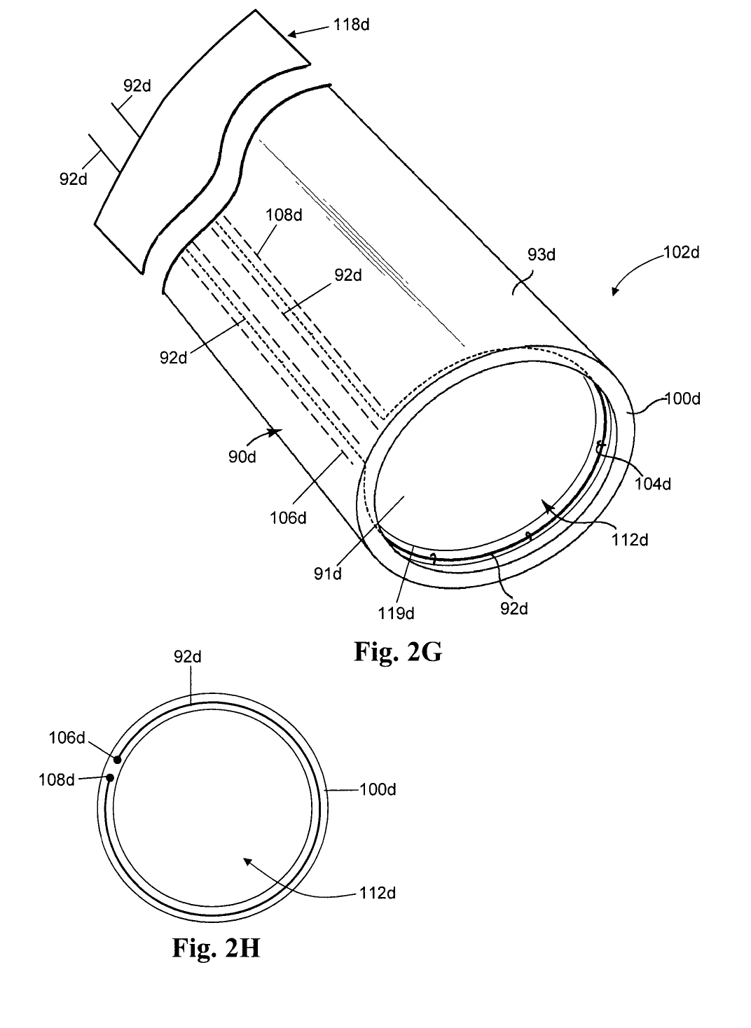

FIG. 2A illustrates one embodiment of a removal instrument 60 in accordance with the present invention. Cutting wire 62 extends along a tubular member 64 and loops around the perimeter 72 of the distal end 66 of the tubular member 64. The distal end 66 defines a distal opening 68 through which a catheter may pass. The wire 62 may be attached to the tubular member 64 by a wire fixing point 70, loop around the perimeter 72 of the distal end 66, and follow a groove 74 to the distal opening 68. The wire 62 may then be placed through an aperture 76 in the tubular member and be strung along the exterior surface 78 of the tubular member 64 so that if a force 80 is applied to the cutting wire 62, the wire fixing point 70 holds one end of the cutting wire 62 stationary so that the cutting wire 62 traverses the distal opening 68 and through a catheter if present.

The cutting wire 62 may be made of any suitable material such as, for example, stainless steel, nickel-titanium alloys or NITINOL alloys. The cutting wire 62 may optionally be heated in order to better cut through a catheter extending through the distal opening 68. In one embodiment, the cutting wire 62 is heated via electricity. The wire fixing point 70 may hold the cutting wire 62 stationary through any suitable mechanism. For example, in one embodiment the wire fixing point 70 may be an adhesive that adheres the cutting wire 62 to the tubular member 64. In another embodiment, the wire fixing point 70 may be a structure that pinches the cutting wire 62. Other suitable wire fixing points 70 may also be used.

FIG. 2B illustrates another embodiment of a removal instrument 90 which also utilizes a cutting wire 92 that extends along a tubular member 93. The cutting wire 92 may be attached to the tubular member 93 at a wire fixing point 98, include a first loop 94 that extends around the perimeter 100 of the distal end 102 of the tubular member and movably engages with the tubular member 93 at a wire fastening point 104. The cutting wire 92 may then extend through the first aperture 106 to a second aperture 108 and out an exterior surface 110 of the tubular member 93. The cutting wire 92 may move relative to the wire fastening point 104.

In these embodiments, the wire fastening point 104 may be a hook or a loop that holds the cutting wire 92 next to the desired location on the tubular member 93, such as the perimeter 100 or the inner surface. The wire fastening point 104 may be located at any suitable point on the perimeter 100. The proximity of the wire fastening point 104 to the wire fixing point 98 will vary the distance across a distal opening 112 that the cutting wire 92 will pass.

The removal instrument 90 cuts the catheter by moving the loop 94 across the distal opening 112 defined by the perimeter 100 of the tubular member 93. In this embodiment, a force 114 may be applied to the cutting wire 92 at the proximal end of the tubular member (not shown) causing the loop 94 to close and move across at least a portion of the distal opening 112 thus cutting the catheter. In some embodiments, the loop 94 may not pass across the entire distal opening 112 due to the proximity of the wire fastening point 104 to the wire fixing point 98. The tubular member 93 may optionally be rotated around the catheter to completely cut through the catheter in this embodiment, or in any other embodiment where the catheter is not fully cut by the cutting wire (or blade as described below).

FIG. 2C illustrates an embodiment of a removal instrument 90a which is similar to the embodiment shown in FIG. 2B and described above. In this embodiment, the axial member is formed of a rod 116 and a ring 117, rather than a tubular member 93 illustrated in FIG. 2B. In this embodiment, the cutting wire 92a may be attached to the ring 117 at a wire fixing point 98a, include a loop 94a that extends around the perimeter 100a of the ring 117 and movably engages with the ring 117 at a wire fastening point 104a. The cutting wire 92a may then extend through the rod 116 to the proximal end 118 of the rod 116.

FIG. 2D illustrates another embodiment of a removal instrument 90b similar to the embodiment illustrated in FIG. 2B, however in this embodiment, the cutting wire 92b does not extend to the perimeter 100b of the tubular member 93b, but to a location on the inner surface of the tubular member 93b. The cutting wire 92b may form loop around the inner surface of the tubular member 93b by using wire fastening points 104b, such as those described above and other devices such as adhesive, which hold the cutting wire 92b adjacent the inner surface of the tubular member 93b such that a loop is formed. The cutting wire 92b is also attached to the tubular member 93b at a wire fixing point 98b.

The removal instrument 90b cuts a catheter or other extension by moving the cutting wire 92b across the distal opening 112b which is defined by the inner diameter of the tubular member 93b. Similar to the previous embodiments, a force may be applied to the cutting wire 92b at the proximal end of the tubular member 93b causing the cutting wire 92b to break away from the wire fastening points 104b and move across at least a portion of the distal opening 112b thus cutting the extension. The tubular member 93b may optionally be rotated around the catheter to completely cut through the extension.

FIGS. 2E and 2F illustrate another embodiment of a removal instrument 90c similar to the embodiment illustrated in FIG. 2B. In this embodiment, however, a wire fixing point 98c and a wire fastening point 104c are positioned on the tubular member 93c such that a cutting wire 92c forms an angle with respect to the plane of the perimeter 100c. As illustrated, the wire fixing point 98c may be located on the perimeter 100c of the tubular member 93c while the wire fastening point 104c, which is formed of an aperture 108c in the illustrated embodiment, may be located proximal to the perimeter 100c. However, in other embodiments, not illustrated, the wire fixing point may be located on the inner surface of the tubular member proximal to the perimeter. In still further embodiments, not illustrated, the perimeter may be angled with respect to the longitudinal axis of the tubular member such that the wire fixing point and the wire fastening point may be both located on the perimeter even if the wire fastening point is proximal to the wire fixing point.

FIG. 2G illustrates another embodiment of a removal instrument 90d similar to the embodiment illustrated in FIG. 2D. In this embodiment, however, the cutting wire 92d extends from the distal end 102d to the proximal end 118d of the tubular member 93d through channels 106d, 108d that are located in the tubular member 93d. In one embodiment, illustrated in FIG. 2G, the channels 106d, 108d may open on the inner surface 91d of the tubular member 93d, such that the cutting wire 92d forms a loop around the inner surface 91d. In an alternative embodiment, illustrated in FIG. 2H, the channels 106d, 108d may open on the perimeter 100d of the tubular member 93d such that the cutting wire 92d forms a loop either around the perimeter 100d or the inner surface 91d. The tubular member 93d may also include a groove 119d in which the cutting wire 92d may be seated. Alternatively, as shown in FIG. 2G, the cutting wire 92d may also be held with wire fastening points 104d, such as those described above.

The removal instrument 90d cuts a catheter or other extension by moving the cutting wire 92d across the distal opening 112d which is defined either by the inner diameter of the tubular member 93d, as in FIG. 2G, or the perimeter 100d, as in FIG. 2H. Similar to the previous embodiments, a force may be applied to the cutting wire 92d at the proximal end 118d of the tubular member 93d causing the cutting wire 92d to release or break away from the wire fastening points 104d and move across at least a portion of the distal opening 112d thus cutting the extension. The tubular member 93d may optionally be rotated around the catheter to completely cut through the extension. Heat or mechanical agitation may also be applied to the cutting wire 92d to facilitate cutting of the catheter or extension.

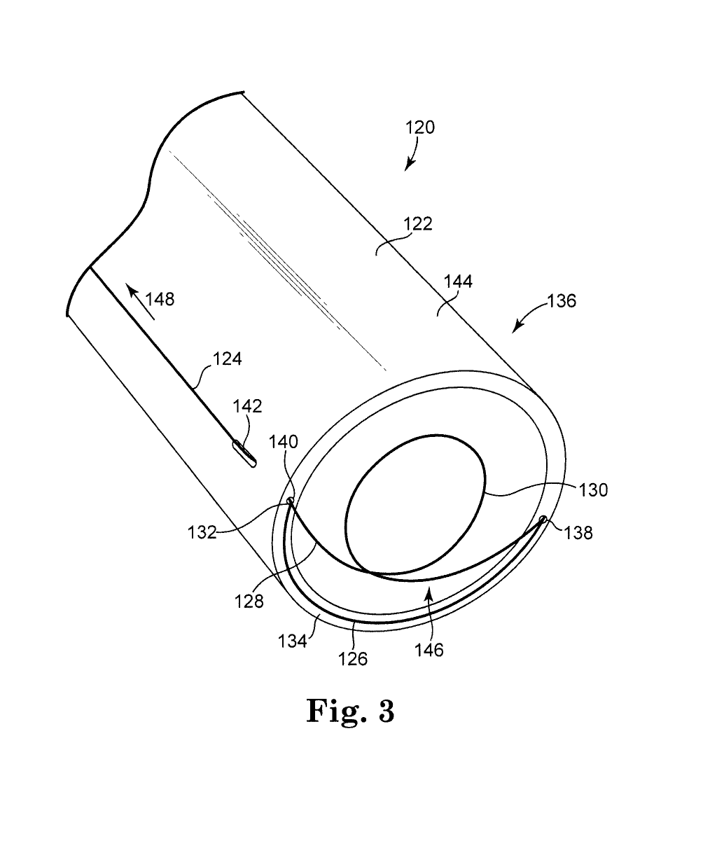

FIG. 3 illustrates another embodiment of a removal instrument 120 which includes a tubular member 122 and a cutting wire 124. The cutting wire 124 also includes a first loop 126 and a second loop 128 that forms a concentric circle 130. The cutting wire 124 may be attached to the tubular member 122 at a wire fixing point 132, extend around a portion of the perimeter 134 of the distal end 136 of the tubular member 122 to a wire fastening point 138 to form the first loop 126. The cutting wire 124 may then extend back around the perimeter 134 to a first aperture 140 to form the second loop 128. The second loop 128 may be twisted one or more times to form the concentric circle 130. In this embodiment, the concentric circle 130 may completely surround a catheter inserted through the tubular member 122. The cutting wire 124 may extend through the first aperture 140 to a second aperture 142 and contact the exterior surface 144 of the tubular member 122. The distal end 136 defines a distal opening 146 across which the concentric circle 130 passes when a force 148 is applied to the cutting wire 124.

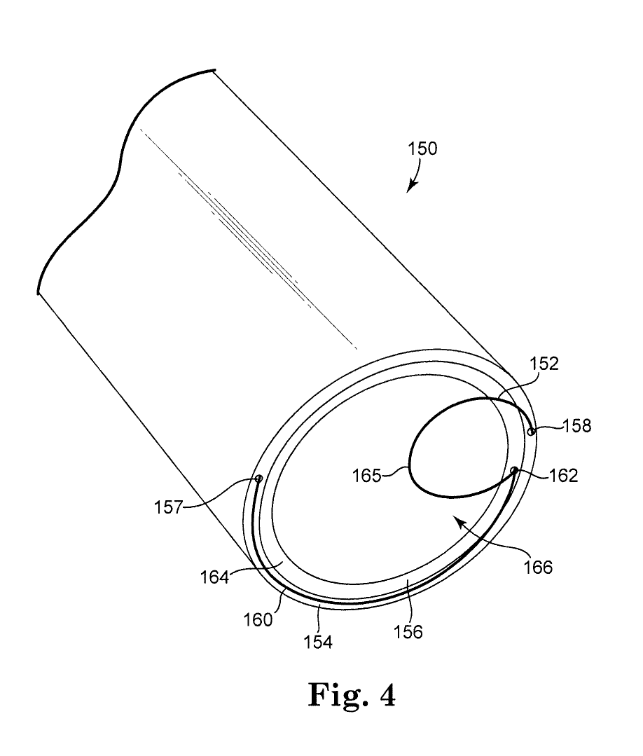

FIG. 4 illustrates another embodiment of a removal instrument 150 that utilizes a cutting wire 152, an outer tubular member 154, and an inner tubular member 156. The cutting wire 152 is attached to a first fixing point 157 and a second fixing point 158, each on the perimeter 160 of the outer tubular member 154. The cutting wire 152 is also moveably fastened to the inner tubular member 156 at a wire fastening point 162 on the perimeter 164 of the inner tubular member 154 and forms a cutting loop 165. The perimeters 160, 164 define a distal opening 166 through which a catheter may be placed. The removal instrument 160 functions by rotating outer tubular member 154 and/or the inner tubular member 156 such that the wire fastening point 162 moves past either the first or second fixed end 156, 158 so that the cutting loop 165 moves across the distal opening 166 and cuts the catheter. In one embodiment, the outer tubular member 154 is rotated clockwise while the inner tubular member 156 remains stationary. In another embodiment, the inner tubular member 156 is rotated counter-clockwise while the outer tubular member 154 remains stationary. In still another embodiment the outer tubular member 154 is rotated clockwise and the inner tubular member 156 is rotated counter-clockwise.

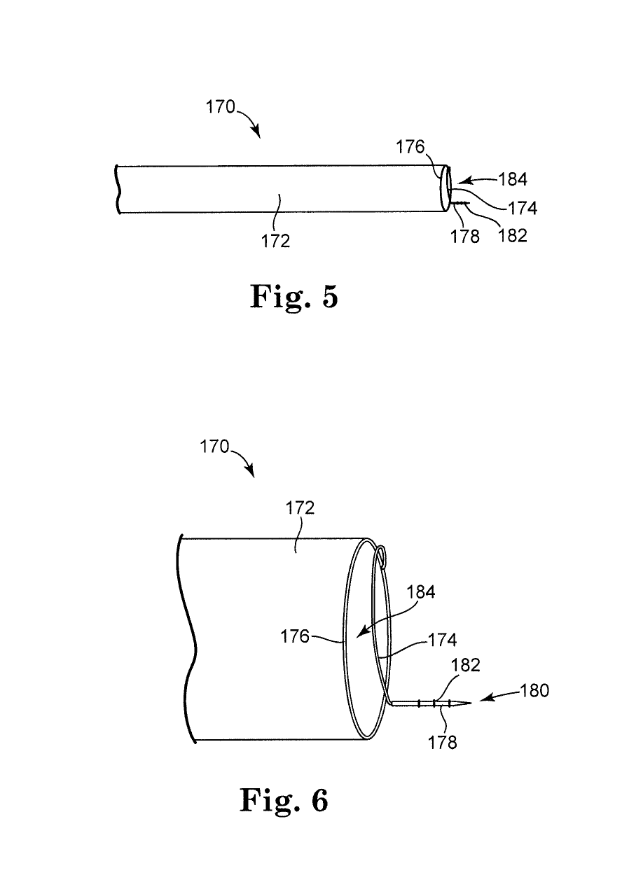

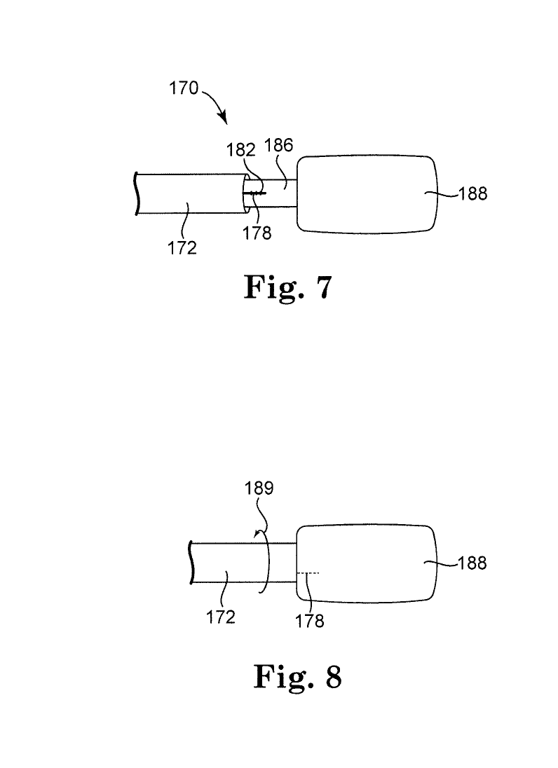

FIGS. 5-8 illustrate another embodiment of a removal instrument 170. Referring to FIGS. 5-6, the removal instrument 170 includes a tubular member 172 and a cutting wire 174 attached to the perimeter 176 of the tubular member 172. Attached to the cutting wire 174 is a dart 178. The dart 178 may optionally include a sharp end 180 (as shown in FIG. 6) and barbs 182. The perimeter 176 defines a distal opening 184.

The removal instrument 170 functions by advancing the tubular member 172 over a catheter 186 attached to a nucleus prosthesis 188 until the dart 178 is anchored in the nucleus prosthesis 188. The sharp point 180 may allow the dart 178 to be easily inserted into the nucleus prosthesis 188 while the barbs 182 may help to prevent the dart 178 from dislodging from the nucleus prosthesis 188. Once the dart 178 is anchored into the nucleus prosthesis 188, a rotational force 189, either clockwise or counter-clockwise, may be applied to the tubular member 172 to move the cutting wire 174 across the distal opening 184 and thus cutting the catheter 186.

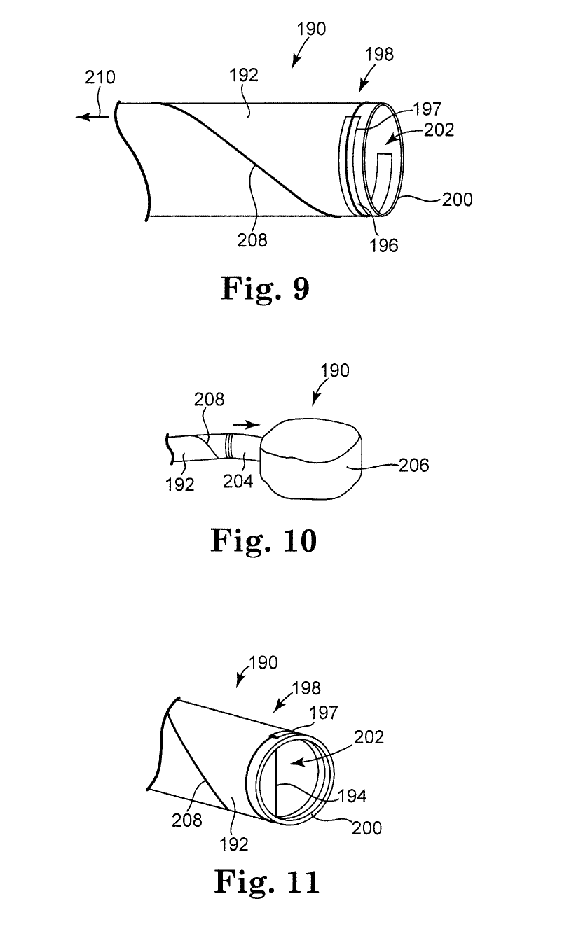

FIGS. 9-11 illustrate another embodiment of a removal instrument 190 that includes a tubular member 192, a cutting wire 194, and a seal 196 that covers a cleft 197 in a distal end 198 of the tubular member 192. The distal end 198 includes a perimeter 200 which defines a distal opening 202. The seal 196 keeps the cutting wire 194 from moving into the distal opening 202 so that the removal instrument 190 can be slid over a catheter 204 that is connected to a nucleus prosthesis 206 without interference by the cutting wire 194. The seal 196 may be made of any suitable material that is capable of keeping the cutting wire 194 from moving into the distal opening 202 but can also be easily broken if a force 210 is applied to the cutting wire. Examples of suitable materials for the seal 196 include paper, and polymeric material. The tubular member 192 may also include a groove 208 that extends from the proximal end of the tubular member (not shown) to the distal end 198 and houses the cutting wire 194.

The removal instrument 190 functions by applying a force 210 to the cutting wire 194. This force 210 creates tension in the cutting wire 194 which breaks the seal 196 covering the cleft 197. Once the seal 196 is broke, the cutting wire 194 is free to move across a portion of the distal opening 202 and partially cut the catheter 204. FIG. 11 shows the position of the cutting wire 194 after the seal 196 is broken and without the catheter 204 present. The distance that the cutting wire 194 is able to move across the distal opening 202 depends upon the size of the cleft 197. In the illustrated embodiment, the cleft 197 extends around more than half of the circumference of the distal opening 202. In other embodiments, the cleft 197 may extend farther around the circumference of the distal opening 202. To completely cut the catheter 204, the removal instrument 190 is rotated either clockwise or counter-clockwise around the catheter 204.

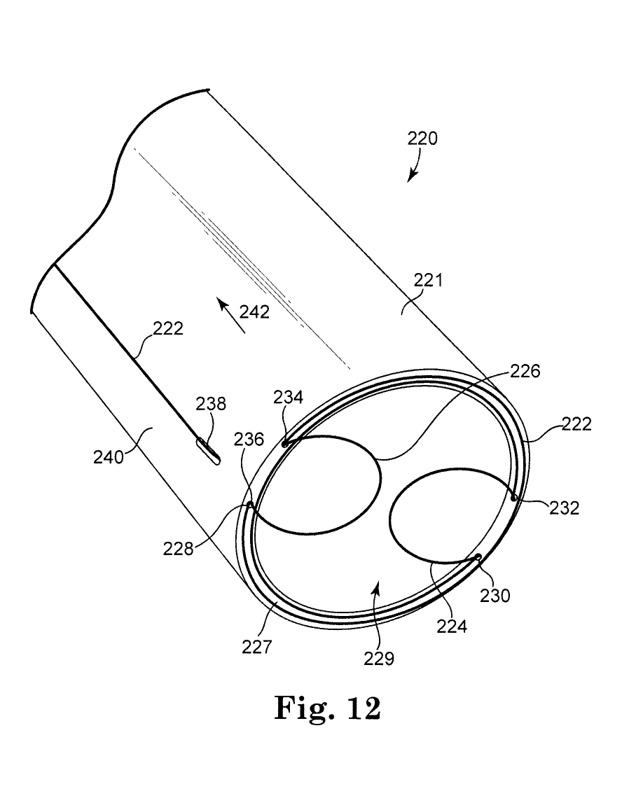

FIG. 12 illustrates another embodiment of a removal instrument 220 which includes a tubular member 221 and a cutting wire 222 that forms a first cutting loop 224 and a second cutting loop 226. The perimeter 227 of the tubular member 221 defines a distal opening 229 and includes a wire fixing point 228, a first fastening point 230, a second fastening point 232, a third fastening point 234, and a first aperture 236 through which the cutting wire 222 is threaded. The tubular member 221 may also include a second aperture 238 through which the cutting wire 222 may extend to reach the exterior surface 240 of the tubular member 221.

Before the removal instrument 220 is slid over a catheter, or before the catheter is placed through the removal instrument 220, the first and second cutting loops 224, 226 should overlap to the extent that neither blocks the distal opening 229. When the catheter is through the distal opening 229, a force 242 may be applied to the cutting wire 222 such that the first and second cutting loops 224, 226 each move across the distal opening 229 in opposite directions and cut through the catheter.

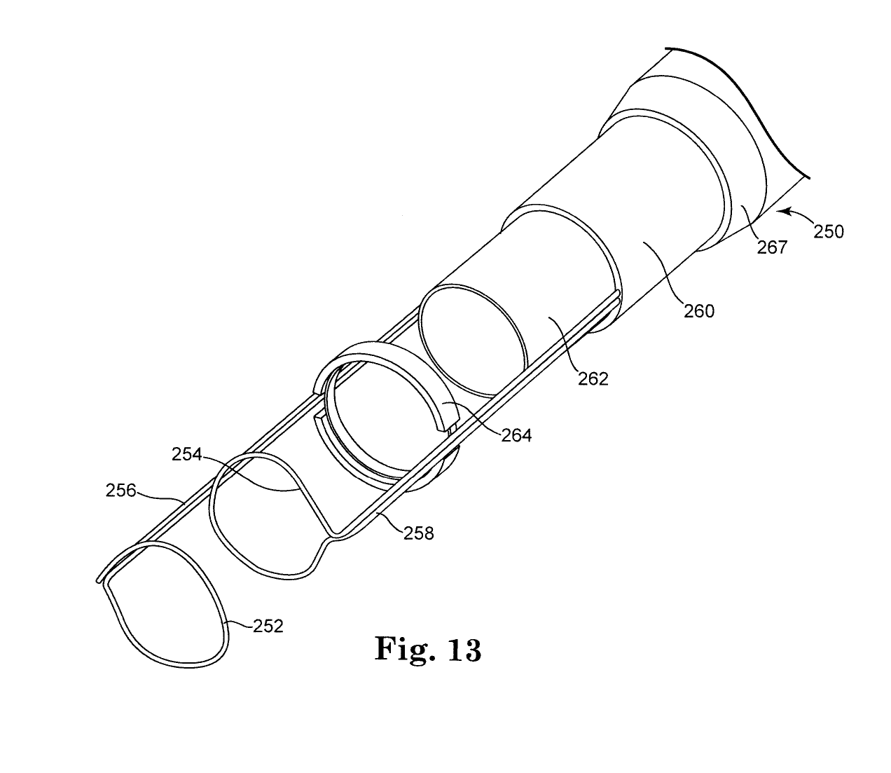

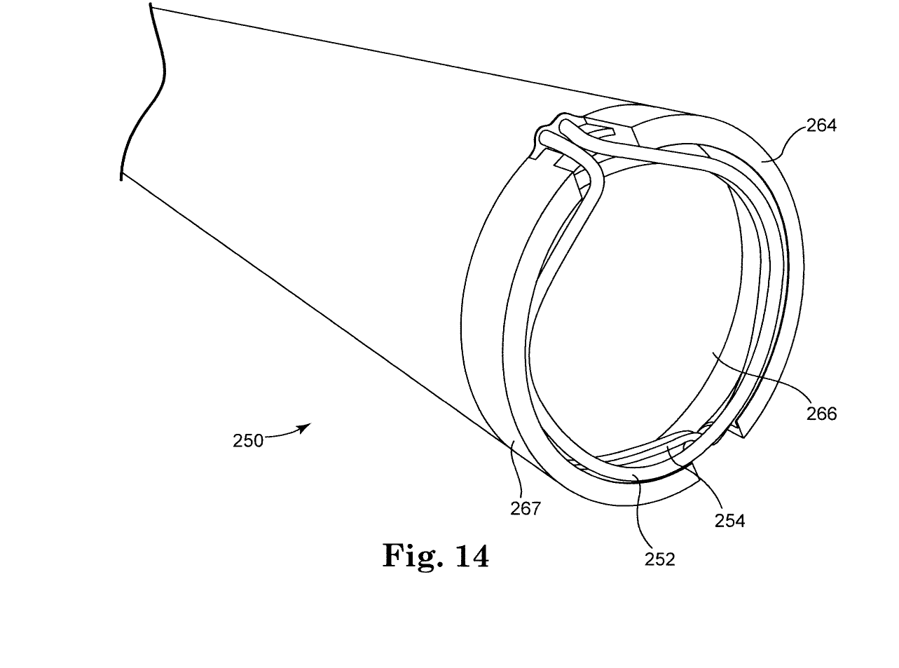

FIGS. 13-15 illustrates a variation of the embodiment shown in FIG. 12. In this embodiment, the removal instrument 250 also includes a first cutting loop 252 and a second cutting loop 254 that are formed of a first cutting wire 256 and a second cutting wire 258. The removal instrument 250 also includes an outer tubular member 260, an inner tubular member 262, and a collar 264 that defines a distal opening 266. The first and second cutting wires 256, 258 may extend to the proximal end of the removal instrument 250 in any suitable location such as between the outer and inner tubular members 260, 262, or between the outer tubular member 262 and the lumen 267.

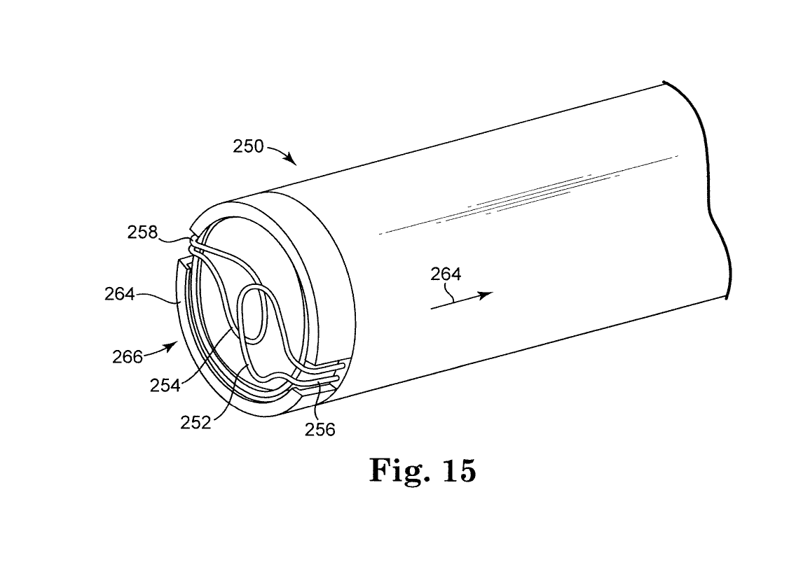

Referring particularly to FIG. 15, the removal instrument 250 functions to cut a catheter in the same manner as the embodiment shown in FIG. 12 except that a force 264 is applied to both the first and second wires 256, 258. This causes the first and second cutting loops 252, 254 to move across the distal opening 266 in opposite directions thus cutting the catheter.

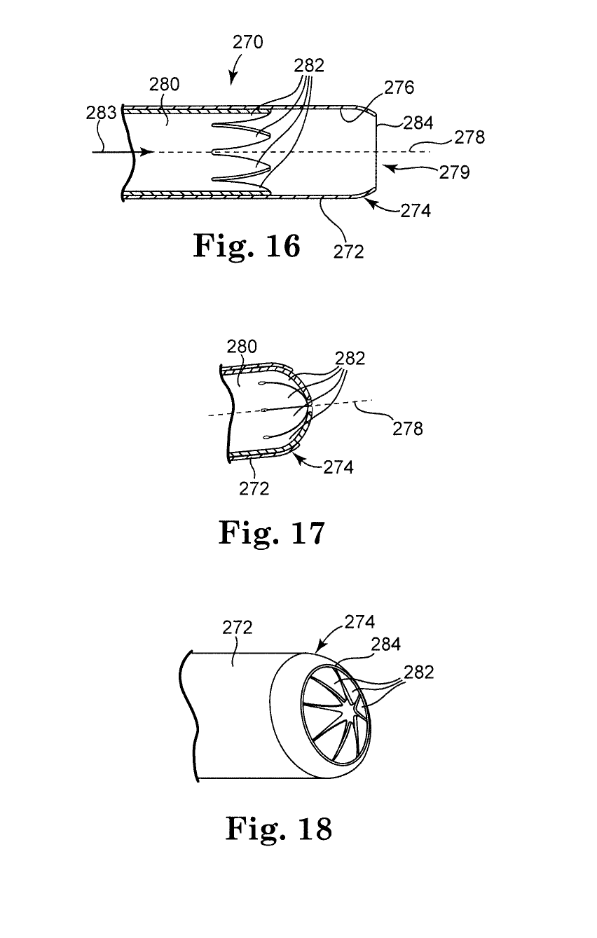

FIGS. 16-21 illustrates another embodiment of a removal instrument 270 which includes an outer tubular member 272 having a distal end 274 and an inner surface 276 that tapers toward a central axis 278 that extends through a distal opening 279 defined by the perimeter 284 at the distal end 274. The removal instrument 270 also includes an inner tubular member 280 that moves through the outer tubular member 272 and includes a plurality of blades 282.

The removal instrument 270 functions by positioning the perimeter 284 of the outer tubular member 272 against the surface of a nucleus prosthesis attached to a catheter, not shown. The inner tubular member 280 is then advanced though the outer tubular member 272 with a force 283 until the blades 282 reach the distal end 274. When the blades 282 reach the distal end 274 of the outer tubular member 272, the tapering inner surface 276 bends the blades 282 toward the central axis 278, into the distal opening 279 and through the catheter.

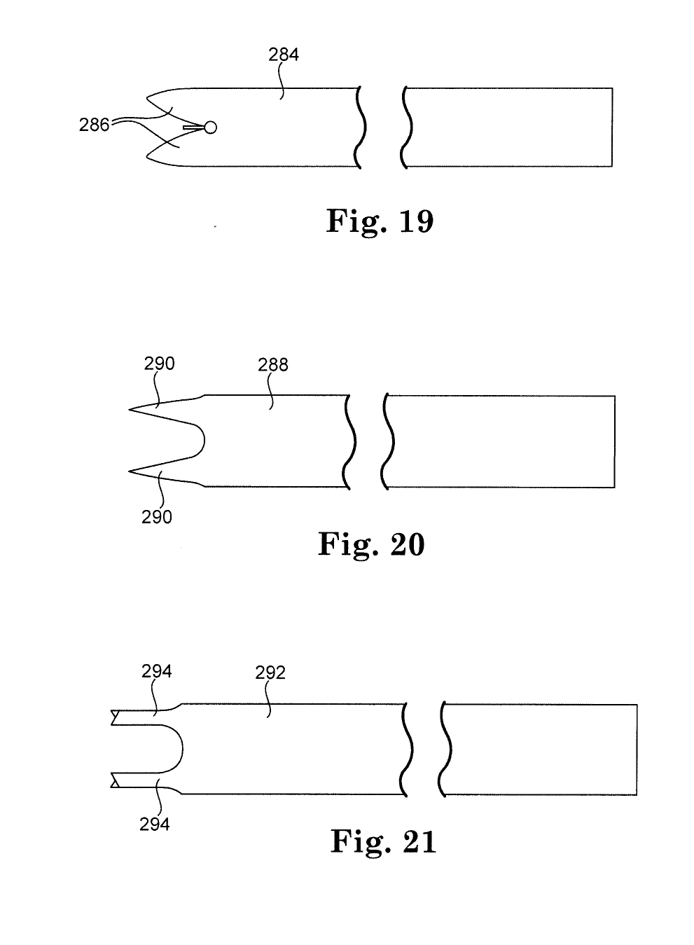

Any suitable number of blades 282 in any suitable shapes or sizes, whether they are pre-bent or flat, may be used. In one embodiment, illustrated in FIGS. 16-18, the inner tubular member 280 includes eight blades 282 each having a parabolic shape. FIG. 19 illustrates another embodiment of an inner tubular member 284 having four blades 286. FIG. 20 illustrates still another embodiment of an inner tubular member 288 having four blades 290 each having a triangular shape. FIG. 21 illustrates another embodiment of a inner tubular member 292 having four flat blades 294.

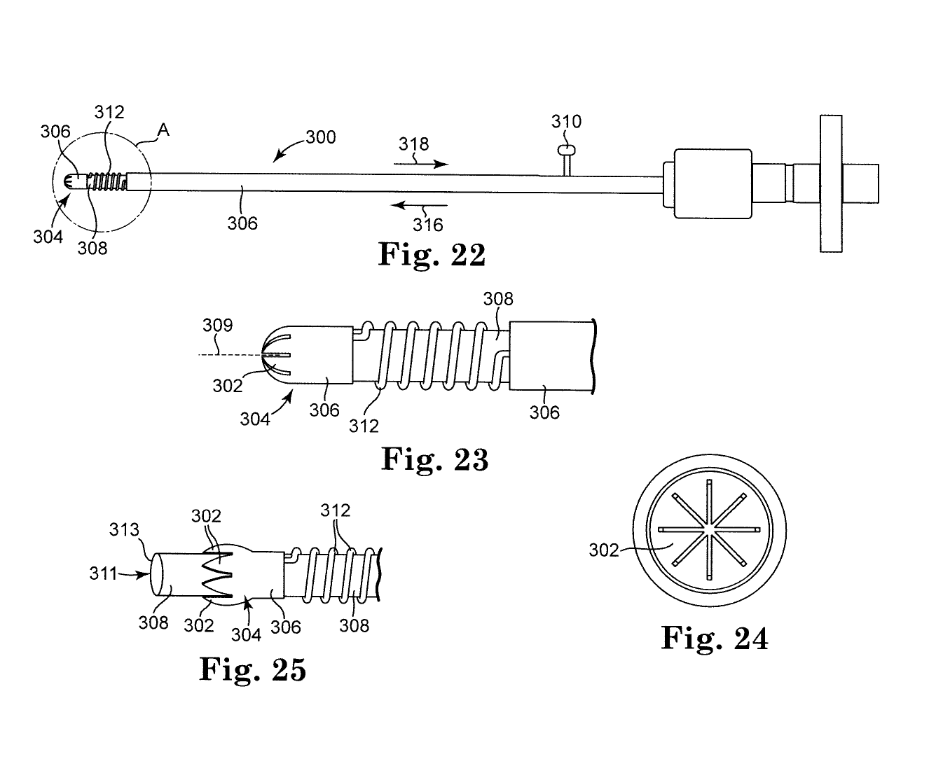

FIGS. 22-25 illustrate another embodiment of a removal instrument 300 in which a plurality of blades 302 are located at the distal end 304 of an outer tubular member 306. The removal instrument 300 also includes an inner tubular member 308 through which a catheter may be placed along a central axis 309. The inner tubular member 308 also includes a distal opening 311 defined by a perimeter 313. In some embodiments, the inner tubular member 308 includes a knob 310 and the outer tubular member 306 includes a window 312 which permits advancement of the inner tubular member 308 relative to the outer tubular member 306. The outer tubular member 306 may optionally include a spring 312 which connects the distal end 304 with the remainder of the outer tubular member 306. The purpose of the spring 312 is to facilitate gradual retraction and replacement of the blades 302, as described below.

In this embodiment, the blades 302 are initially formed curved toward the central axis 309 and covering the distal opening 311. The blades 302 may be formed of a memory-shaped alloy, such as for example Nitinol or spring steel, to facilitate returning the blades 302 to this curved position after they are retracted. Before the catheter is placed through the distal opening 311, the blades 302 may be retracted in a proximal direction 318 to allow the catheter, not shown, through the distal opening 311. To retract the blades 302, and thus open the distal opening 311, the inner tubular member 308 is advanced in a distal direction 316 while the outer tubular member 306 is kept substantially stationary. As the perimeter 313 pushes against the blades 302, the blades 302 retract, as shown in FIG. 25, away from the distal opening 311 so that the catheter may be placed though the distal opening 311. When it is time to cut the catheter, the blades 302 are positioned adjacent the perimeter 313 of the inner tubular member 308 so that when the inner tubular member 308 is retracted in a proximal direction 318, the blades 302 move back toward the central axis 309, thus cutting through the catheter.

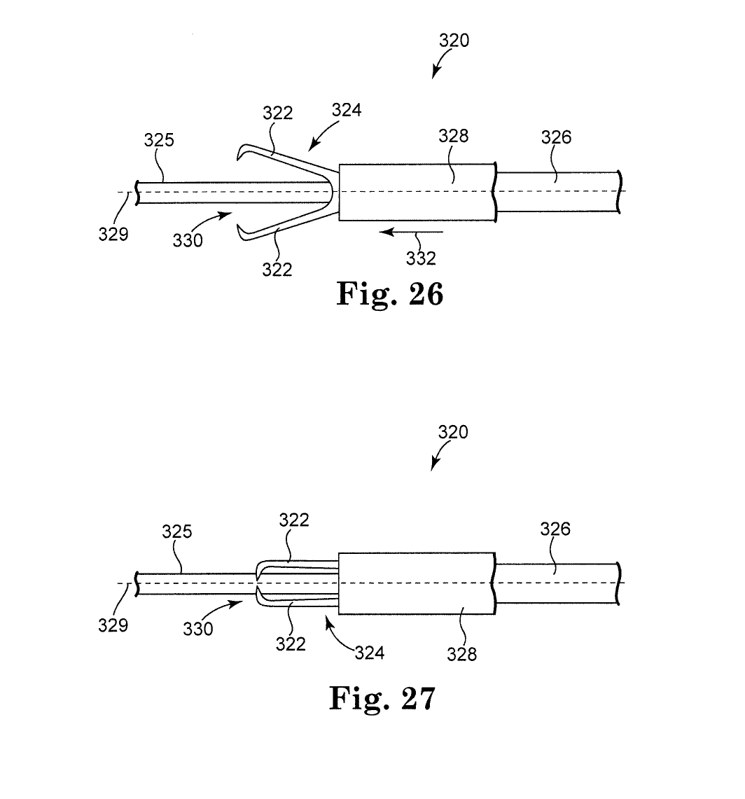

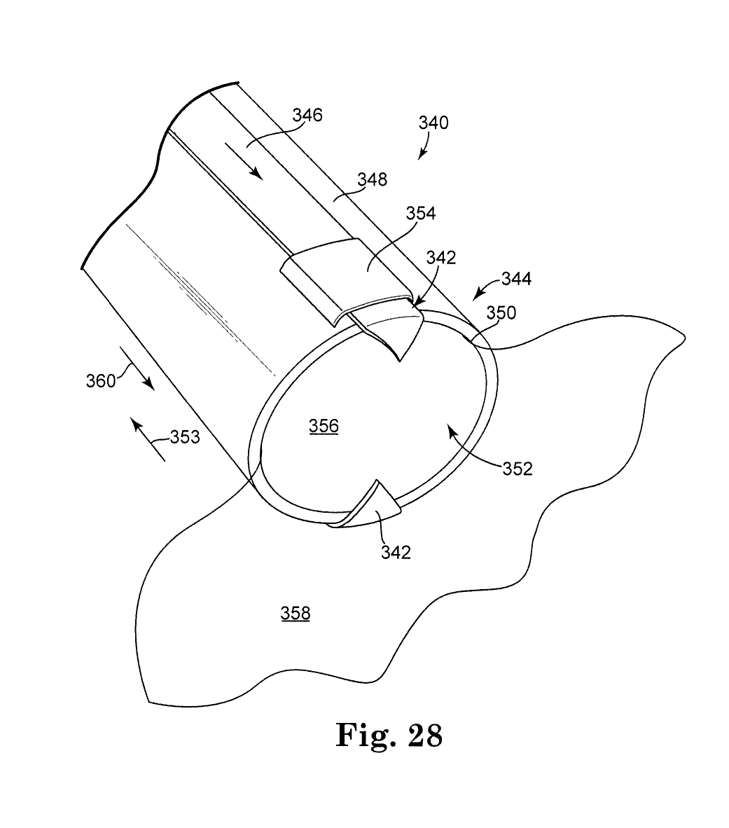

Another embodiment of a removal instrument 320, illustrated in FIGS. 26-27, includes one or more blades 322 at the distal end 324 of an inner tubular member 326 and an outer tubular member 328. A central axis 329 runs down the center of the inner tubular member 326. The blades 322 are initially formed in a retracted position such that they do not block a distal opening 330 in the inner tubular member 326. The removal instrument 320 cuts a catheter 325 that is located in the distal opening 330 by advancing the outer tubular member 328 in a distal direction 332 such that the blades 322 are forced toward the central axis 329 thus at least partially cutting the catheter 325. In the illustrated embodiment, the removal instrument 320 includes two blades 322, however embodiments using one blade or more than one blade may also be used.

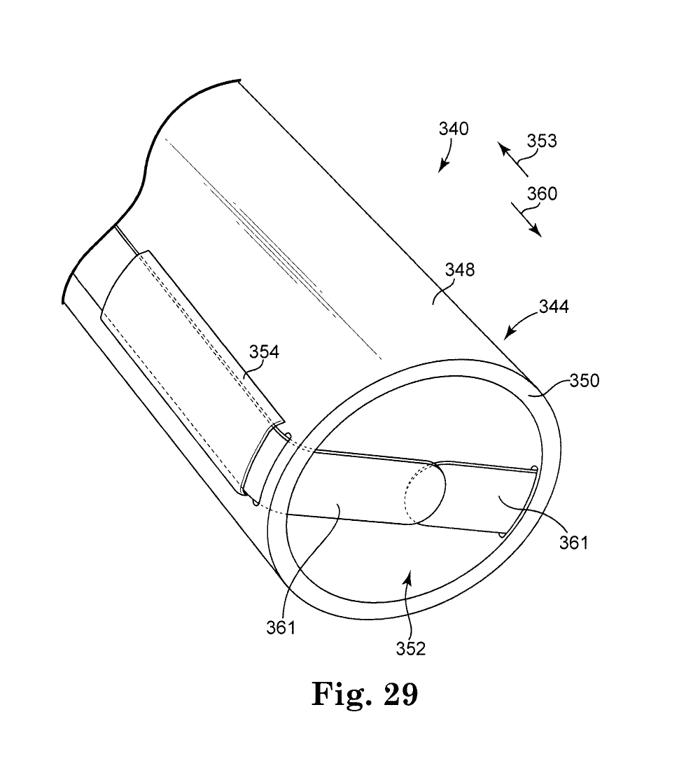

FIGS. 28-29 illustrates an embodiment of a removal instrument 340 in which one or more blades 342 are included at a distal end 344 of one or more blade handles 346. The removal instrument 340 also includes a tubular member 348 which has perimeter 350 that defines a distal opening 352. The tubular member 348 also includes one or more housings 354. This removal instrument 340 functions by retracting the blade handles 346 in a proximal direction 353 which causes the blades 342 to move into the housings 354 and do not cover the distal opening 352. If the catheter 356 is already in place, the removal instrument 340 may be slid over the catheter 356 to the appropriate location for cutting. Alternatively, the catheter 356 and the unexpanded nucleus prosthesis 358 may be inserted through the distal opening 352 after the blades 342 are retracted. When the catheter 356 is ready to be cut, the blade handles 346 are advanced in a distal direction 360, the blades curve around the perimeter 350, move into the distal opening 352 thus at least partially cutting the catheter 356. In the embodiment illustrated in FIG. 28, the blades 342 are not extended so far as to cut through the entire catheter 356. In the embodiment illustrated in FIG. 29, blades 361 extend to the point that they overlap.

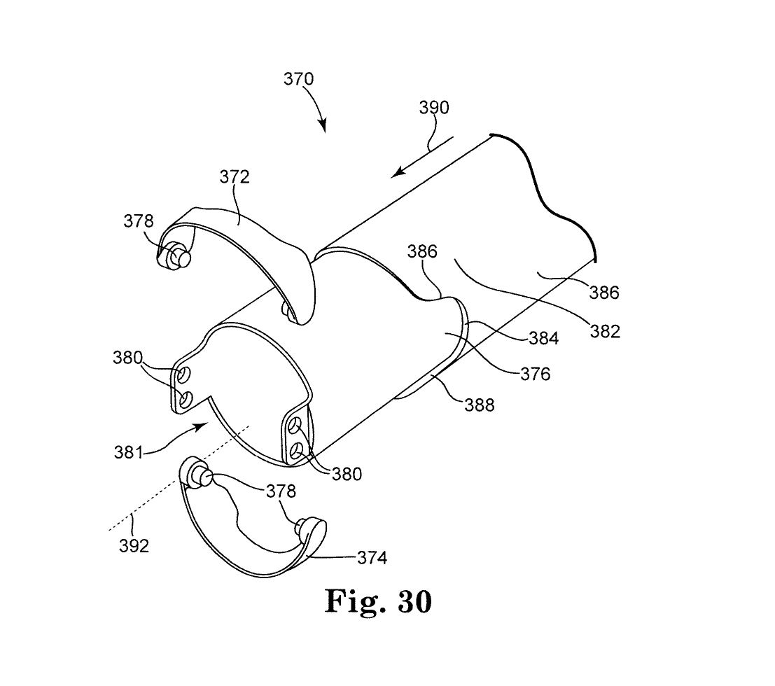

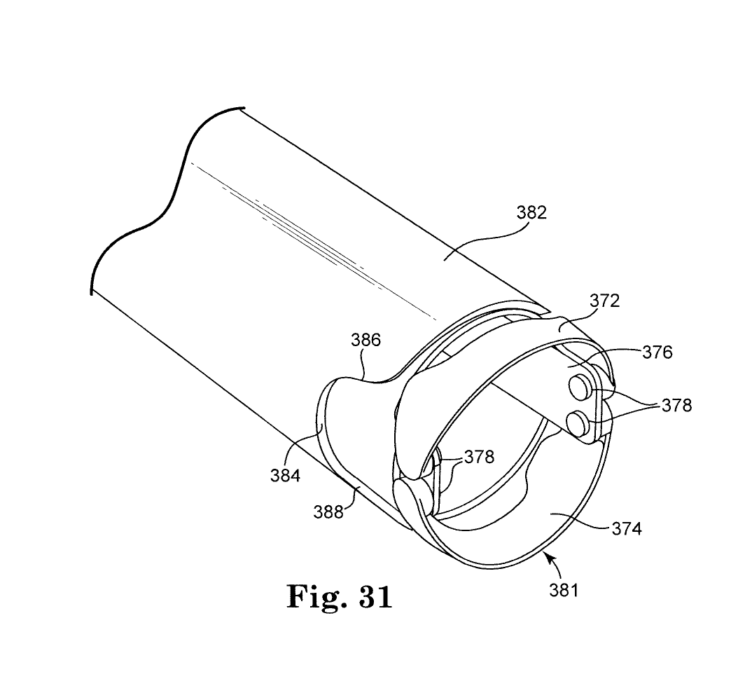

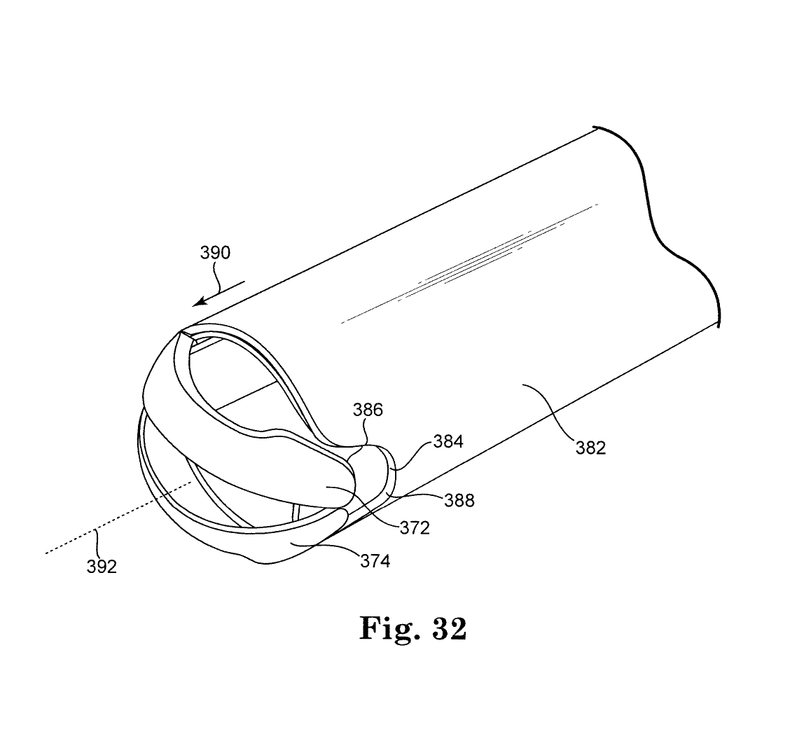

FIGS. 30-32 illustrate another embodiment of a removal instrument 370 which includes a first blade 372 and a second blade 374 each movably attached to an inner tubular member 376. The first and second blades 372, 374 may moveably attach to the inner tubular member 376 in any suitable manner that allows the first and second blades 372, 374 to pivot. For example, as illustrated, each of the first and second blades 372, 374 may include opposing rivets 378 that fit into pivot holes 380 on the inner tubular member 376. The inner tubular member 376 also defines a distal opening 381. The removal instrument 370 also includes an outer tubular member 382 having opposing recesses 384. Each of the recesses 384 include a first surface 386 facing a second surface 388. The removal instrument 370 functions by advancing the outer tubular member 382 in a distal direction 390 toward the first and second blades 372, 374 so that the first and second surfaces 386, 388 contact the first and second blades 372, 374 and push the first and second blades 372, 374 toward a central axis 392. The intervertebral catheter located in the distal opening 381 will then be cut.

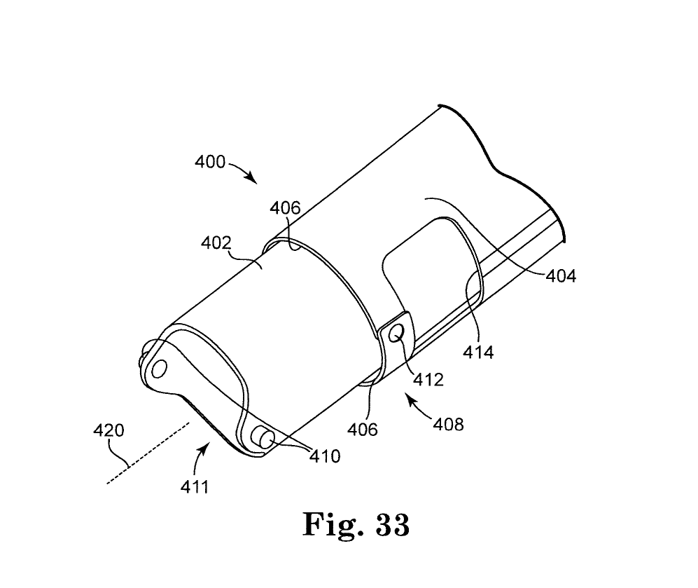

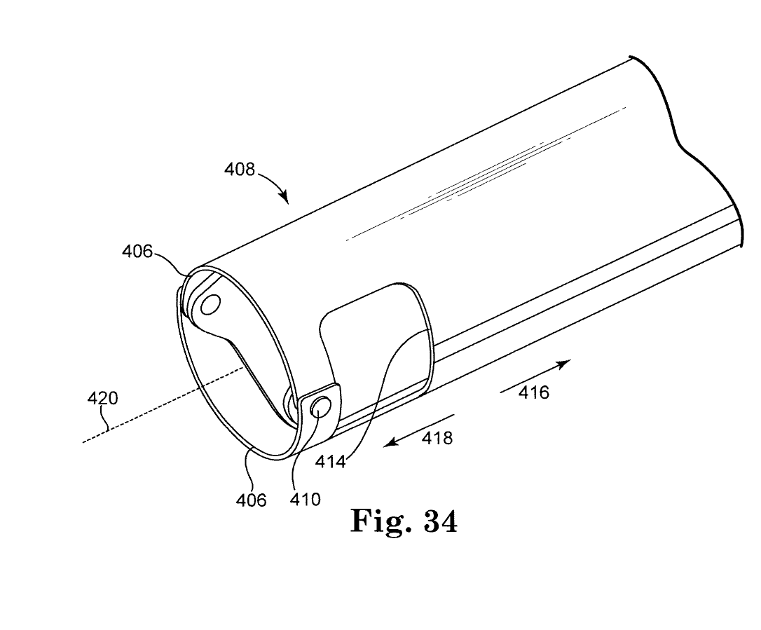

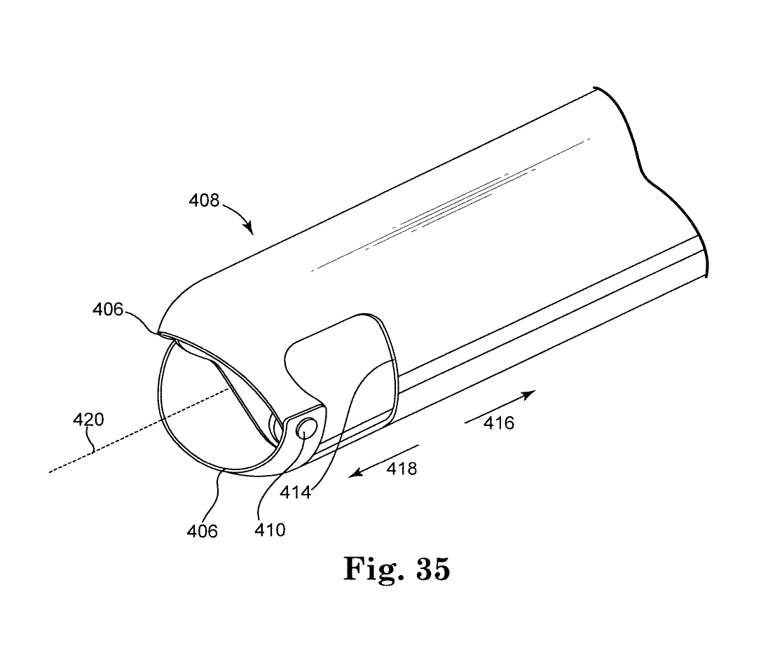

FIGS. 33-35 illustrate an embodiment of a removal instrument 400 which includes an inner tubular member 402 and an outer tubular member 404 with blades 406 located at a distal end 408 of the outer tubular member 404. The outer tubular member 404 pivots with respect to the inner tubular member 402. Any suitable structure may be formed to allow this pivoting, however in the illustrated embodiment, the inner tubular member 402 includes opposing rivets 410 which fit into opposing pivot holes 412 on the outer tubular member 404. The outer tubular member 404 may also include opposing windows 414 which give the rivets 410 space to move with respect to the blades 406 so that the outer tubular member 404 is able to deform, as descried below. The inner tubular member 402 defines a distal opening 411 through which a catheter may be inserted.

When the rivets 410 are held in the pivot holes 412 and the inner tubular member 402 is retracted in a proximal direction 416 or the outer tubular member 404 is advanced in a distal direction 418, the outer tubular member 404 deforms and the blades 406 move toward a central axis 420 located through the inner tubular member 402 thus closing the distal opening 411 and cutting the catheter.

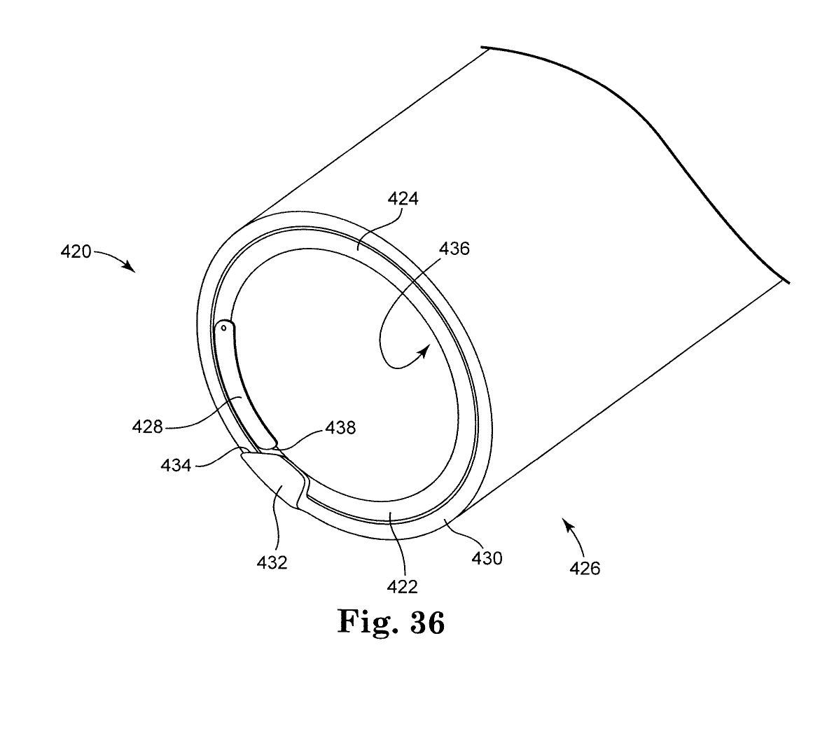

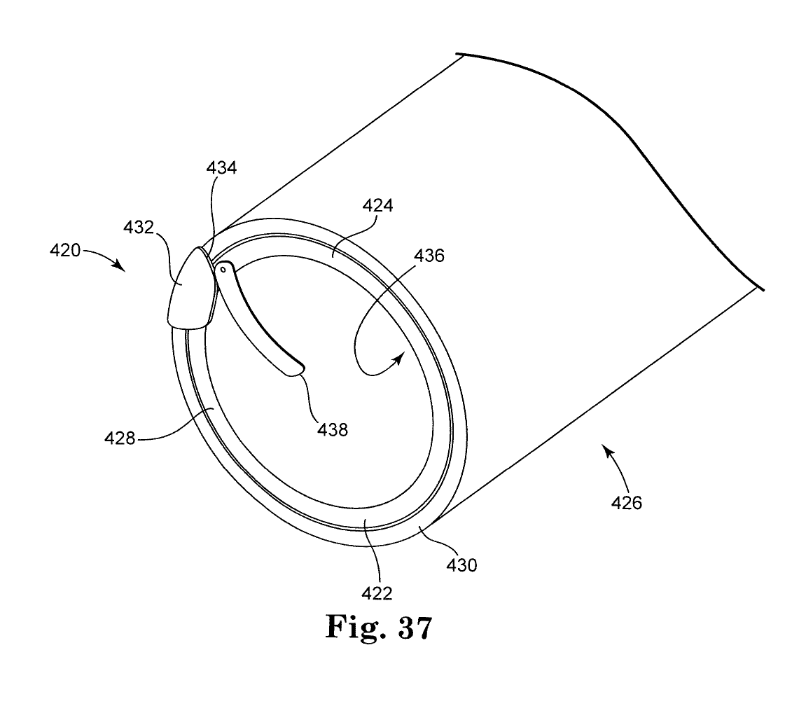

In still another embodiment, illustrated in FIGS. 36-37, a removal instrument 420 includes an inner tubular member 422 having a perimeter 424 at the distal end 426, a blade 428 pivotally attached to the perimeter 424 and an outer tubular member 430 having a wedge 432 with an inclined surface 434. The perimeter 424 defines a distal opening 436 across which the blade 428 moves when the outer tubular member 430 rotates relative to the inner tubular member 422. The blade also includes an tapering surface 438 that tapers in the opposite direction as the inclined surface 434 of the wedge 432. As the inner and outer tubular members 422, 430 move relative to each other, the inclined surface of the 434 contacts the tapering surface 438 and pushes the blade 428 across the distal opening 436 thus cutting the catheter if present. In this embodiment the inner tubular member 422 may be rotated while the outer tubular member 430 remains relatively stationary. In one alternative, the outer tubular member 430 may be rotated while the inner tubular member 422 remains relatively stationary. In another alternative the inner tubular member 422 may be rotated in one direction while the outer tubular member 430 is rotated in the opposite direction. Whether the inner and outer tubular members 422, 430 are rotated clockwise or counter-clockwise will depend upon the shape of the included surface 434 and the tapering surface 438.

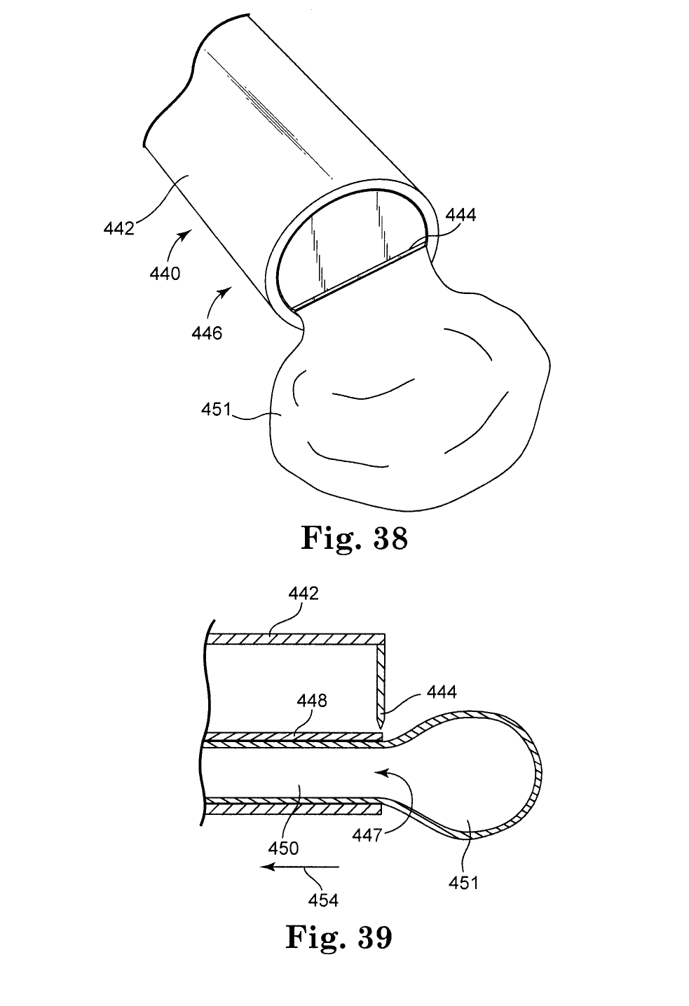

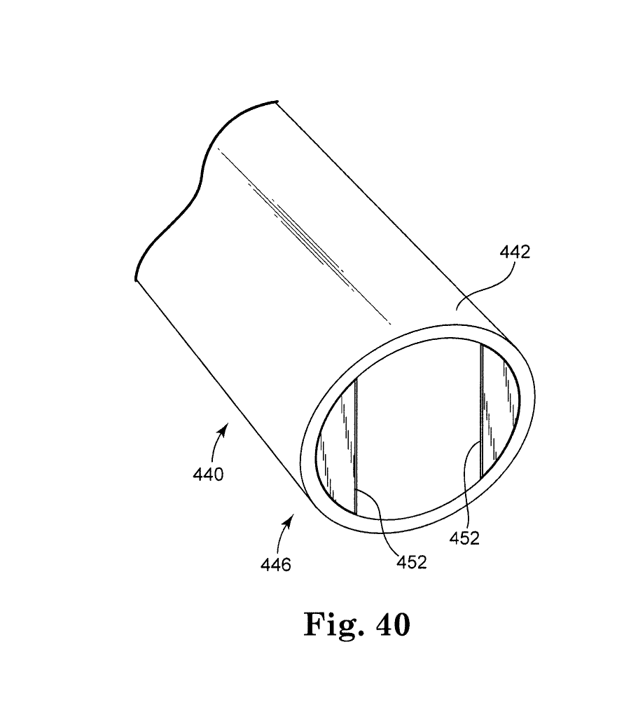

In another embodiment, illustrated in FIGS. 38-39, a removal instrument 440 includes a tubular member 442 having one or more blades 444 at a distal end 446 of the tubular member 442, a distal opening 447, and a blade guard 448. This removal instrument 440 functions by positioning the blade guard 448 to cover the one or more blades 444 while inserted over a catheter 450 through the distal opening 447. When the catheter 450 is ready to be cut, the blade guard 448 may then be retracting in a proximal direction 454 to expose the one or more blades 444 to the catheter 450. The tubular member 442 may then be rotated to in any direction push the one or more blades 444 through a portion of the catheter 450. FIG. 40 illustrates an embodiment similar to the embodiment illustrated in FIGS. 38-39 except that it includes two blades 452 rather than one.

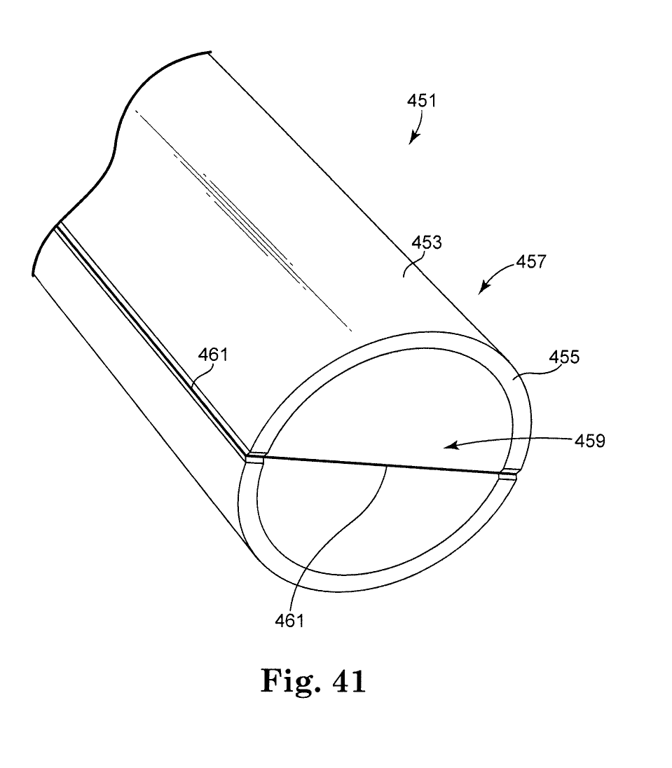

FIG. 41 illustrates another embodiment of a removal instrument 451. The removal instrument 451 includes a tubular member 453 having a perimeter 455 at a distal end 457, a distal opening 459, and a cutting wire 461 that is able to span the distal opening 459. Before advancing the removal instrument 451 over a catheter, the cutting wire 461 may be pulled to the perimeter 455 such that it does not block the distal opening 459. Tape, adhesives, hooks, or other means of attachment may be used to keep the wire adjacent the perimeter 455 or the internal diameter of the tubular member 453, so that it does not block the distal opening 459. When the removal instrument 451 is in the desired position to cut the catheter, the cutting wire 461 may be released from the perimeter 455 so that it moves into the distal opening 459 to cut the catheter.

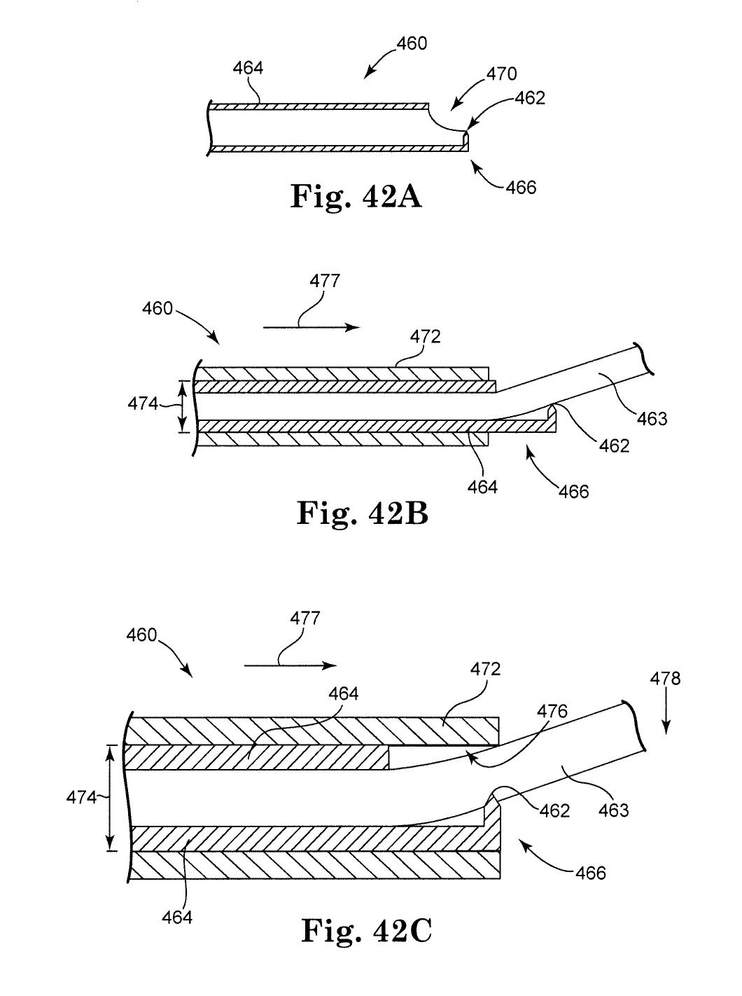

FIGS. 42A-42C illustrate an embodiment of a removal instrument 460 which includes a blade 462 that is capable of positively engaging with a catheter 463. In this embodiment, the removal instrument 460 includes an inner tubular member 464 which includes a distal end 466. The removal instrument 460 also includes a distal opening 470 through which a catheter 463 may be placed. The removal instrument 460 also includes an outer tubular member 472 that includes an inside diameter 474 that allows the inner tubular member 464 fit inside the outer tubular member 472 and an inside surface 476. The removal instrument 460 functions by advancing the inner tubular member 464 over the catheter 463 to a desired cutting point. The catheter 463 will extend out the distal end 466 of the inner tubular member 464. The outer tubular member 472 is then advanced over the inner tubular member 464 and past the distal opening 470 using a first force 477. At this point the inside surface 476 will exert a second force 478 on the catheter 463 that pushes the catheter 463 onto the blade 462 and at least partially cuts the catheter 463. If needed, the outer tubular member 472 may be retracted, the inner tubular member 464 may be rotated and the outer tubular member 472 may be advanced and the inner tubular member 464 may then be rotated down the catheter to cut it until the final cut is achieved. Alternatively, the outer tubular member 472 may be advanced, and the inner tubular member 464 may be rotated down the catheter to cut it, until the final cut is achieved. In such an embodiment, the blade 462 would suitably be angled with respect to the plane of the distal opening 470 in order to facilitate the spiral cutting.

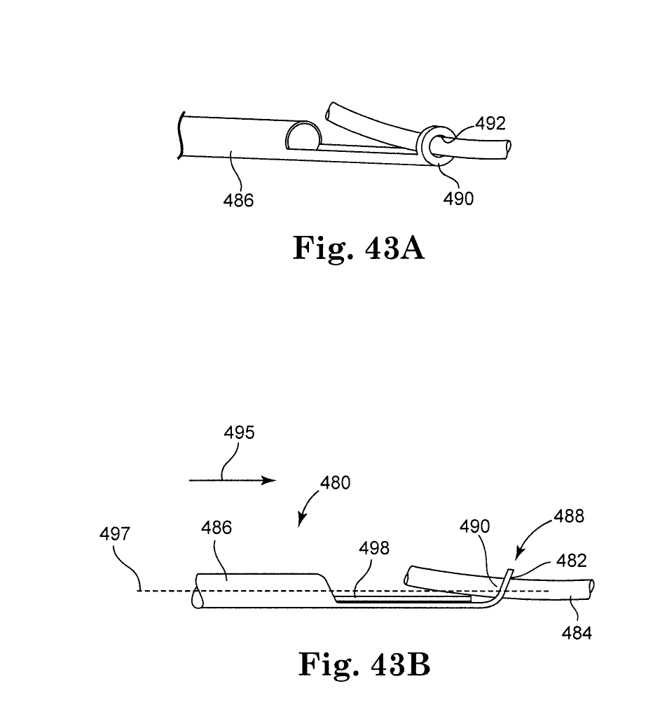

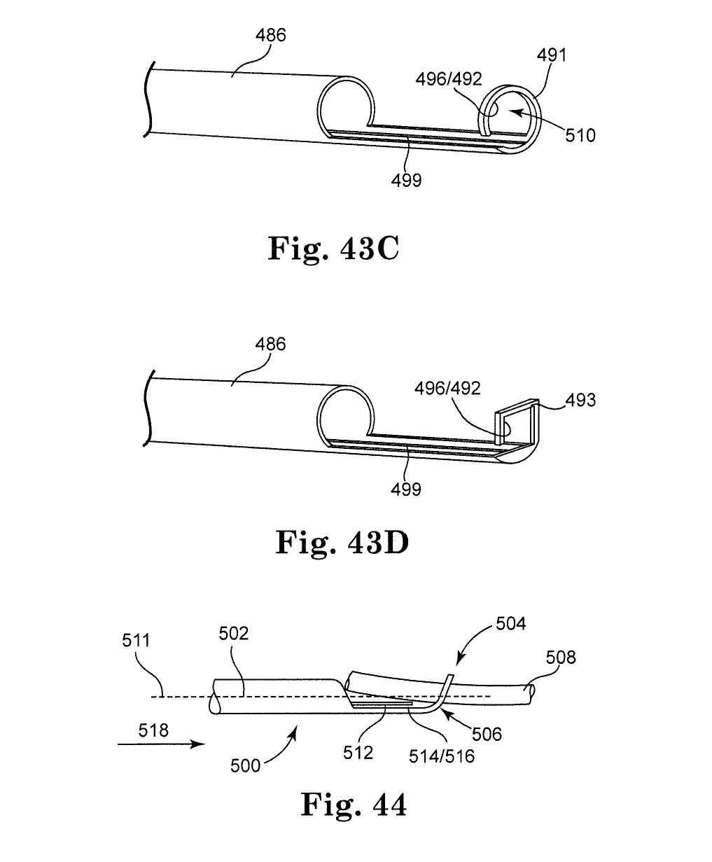

FIGS. 43A-43D also illustrates an embodiment of a removal instrument 480 which includes a cutter 496, such as a blade 482, that is capable of positively engaging with an extension 484. In this embodiment, the removal instrument 480 includes an outer tubular member 486 which includes a distal end 488, and a perimeter 490 having an inner edge 492 that is capable of receiving the extension 484 through a distal opening 510. In this embodiment, the cutter 496, or blade 482, may be positioned on the inner edge 492. The perimeter 490 may be positioned at an angle relative to the longitudinal axis 497 of the outer tubular member 486. The removal instrument 480 also includes an inner axial member 498 which may fit in a channel 499 on the interior portion of the outer tubular member 486. When a force 495 is applied to the inner axial member 498, the extension 484 is pushed toward the cutter 496 or blade 482, on the inner edge 492 of the perimeter 490 to cut the extension 484. If needed, the outer tubular member 486 may be rotated around the extension 484 and the inner axial member 498 may be advanced again to further cut the extension 484.

FIG. 44 also illustrates an embodiment of a removal instrument 500 which includes an outer tubular member 502 having a distal end 504, and a perimeter 506 that is capable of receiving an extension 508 through a distal opening, not shown. In this embodiment, the perimeter 506 may be positioned at an angle relative to the longitudinal axis 511 of the outer tubular member 502. The removal instrument 500 also includes an inner axial member 512 having a distal end 514 and a cutter 516 at the distal end. When a force 518 is applied to the inner axial member 512, the inner axial member 512 is advanced toward the distal opening (not shown) and the cutter 516 engages with and cuts the extension 508 positioned in the distal opening (not shown). In one embodiment, the outer tubular member 502 may also include a channel (not shown) which may guide the inner axial member 512 to the perimeter 506. If needed, the removal instrument 500 may be rotated around the extension 508 and the inner axial member 512 may be advanced again to further cut the extension 508.

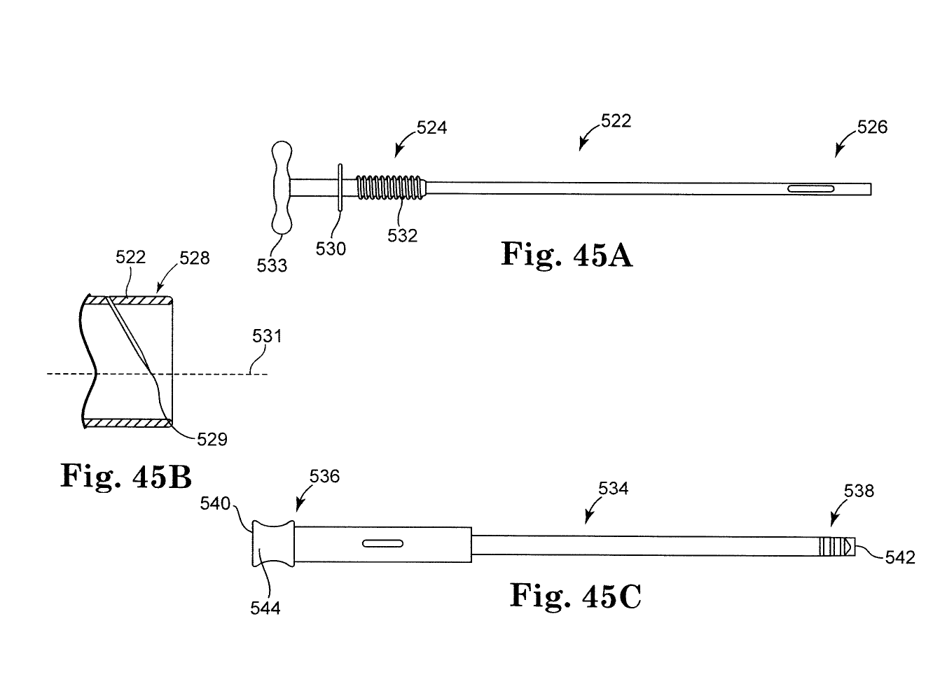

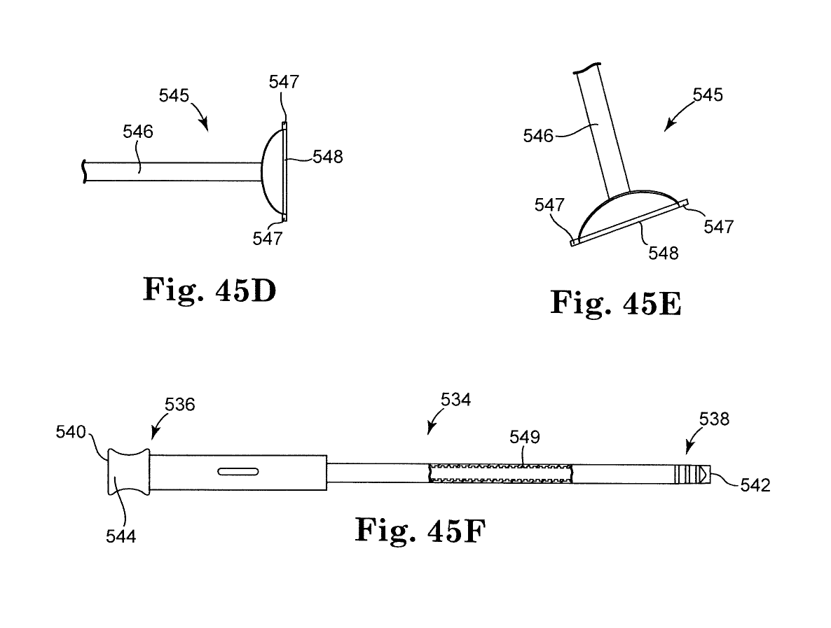

FIGS. 45A-45C illustrates another embodiment of a removal instrument that includes an inner axial member 522 having a proximal end 524, a distal end 526, and a cutter 528 at the distal end 526, more particularly seen in FIG. 45B. The inner axial member 522 also includes a collar 530, a first threaded portion 532, and a handle 533. The cutter 528 may include a blade 529 that is angled from a central axis 531. In the illustrated embodiment, the collar 530 and the first threaded portion 532 are located at the proximal end 524, although in other embodiments they may be located elsewhere on the inner axial member 522. The removal instrument also includes an outer tubular member 534 that includes a proximal end 536, a distal end 538, a proximal surface 540, a distal surface 542, and a second internal threaded portion 544. The removal instrument operates by placing the outer tubular member 534 over an extension, such as a catheter, to the point that the distal surface 542 is at the plane that the user would like to remove the extension. The inner axial member 522 is then placed inside of the outer tubular member 534 so that the cutter 528 contacts the exposed end of the extension. Using the handle 533, the inner axial member 522 is rotated. The first threaded portion 532 on the inner axial member 522 may then engage with the second threaded portion 544. These threaded portions 532, 544 provides uniformity to the pitch of the cutting, even among different users applying different amounts of pressure or torque to the inner axial member 522. The cutter 528, or blade 529 if included, makes a spiral cut down the length of the extension. The collar 530 is shaped such that when the collar 530 contacts the proximal surface 540 of the outer tubular member 534, the inner axial member 522 is prevented from advancing further into the outer tubular member 534. As a result, the cutter 528 cannot advance past the plane of the distal surface 542 of the outer tubular member 534.

FIGS. 45D-45F illustrate a variation of the embodiment of 45A-45C. In this embodiment, the inner axial member 545 may include a rod 546, a blade 548, and at least one thread engaging member 547. In the illustrated embodiment, the inner axial member 545 includes two thread engaging members 547. The inner axial member 545 fits within the outer tubular member 534 described above. In this embodiment, however, the outer tubular member 534 may include a threads 549 on the inside surface of the outer tubular member 534. The thread engaging members 547 engage with the threads 549 of the outer tubular member 534 such that when torque is applied to the rod 546, the blade 548 rotates while advancing down the shaft of the outer tubular member 534 cutting any extension in the outer tubular member 534. The torque applied to the inner axial members 522, 545, may be applied using a mechanized device, such as a drill.

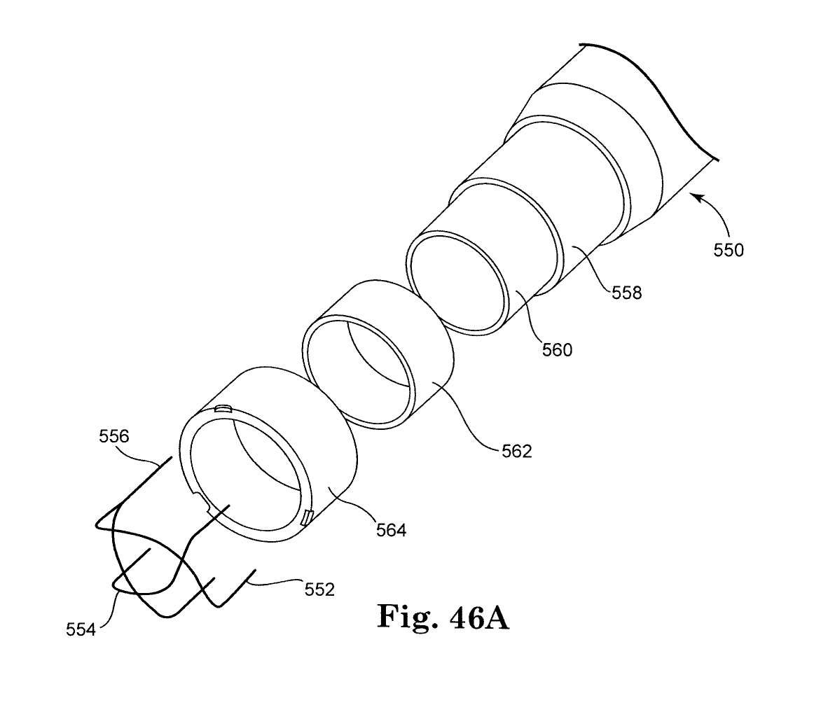

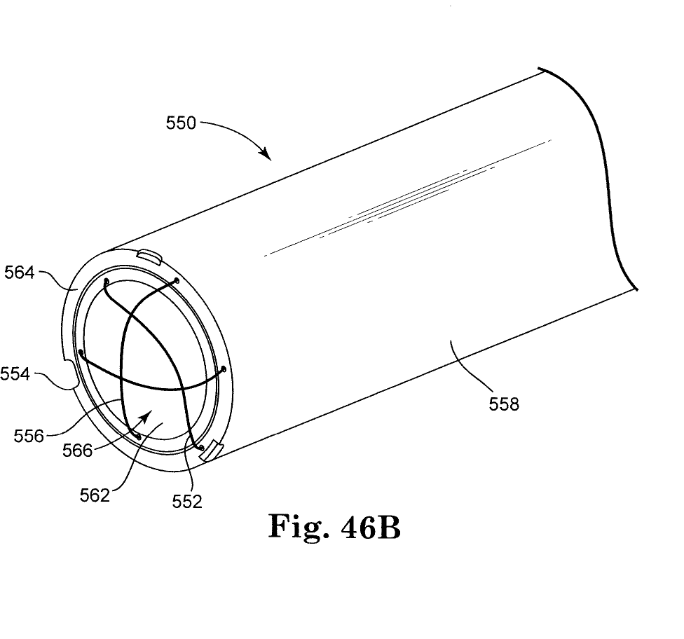

FIGS. 46A-46B illustrate another embodiment of a removal instrument 550. In this embodiment, the removal instrument 550 also includes a first cutting wire 552, a second cutting wire 554, and a third cutting wire 556. The removal instrument 550 also includes an outer tubular member 558, an inner tubular member 560, a first collar 562, which may be attached to the inner tubular member 560, and a second collar 564, which may be attached to the outer tubular member 558, that defines a distal opening 566. One end of each of the first, second and third cutting wires 552, 554, 556 may be attached to the inner tubular member 560 or the first collar 562. The other end of the first, second and third cutting wires 552, 554, 556 may be attached to the outer tubular member 558 or the second collar 564.

Referring particularly to FIG. 46B, the removal instrument 550 functions by rotating the inner tubular member 560 while rotating or keeping fixed the outer tubular member 558. This causes the first, second and third cutting wires 552, 554, 556 to move across the distal opening 566.

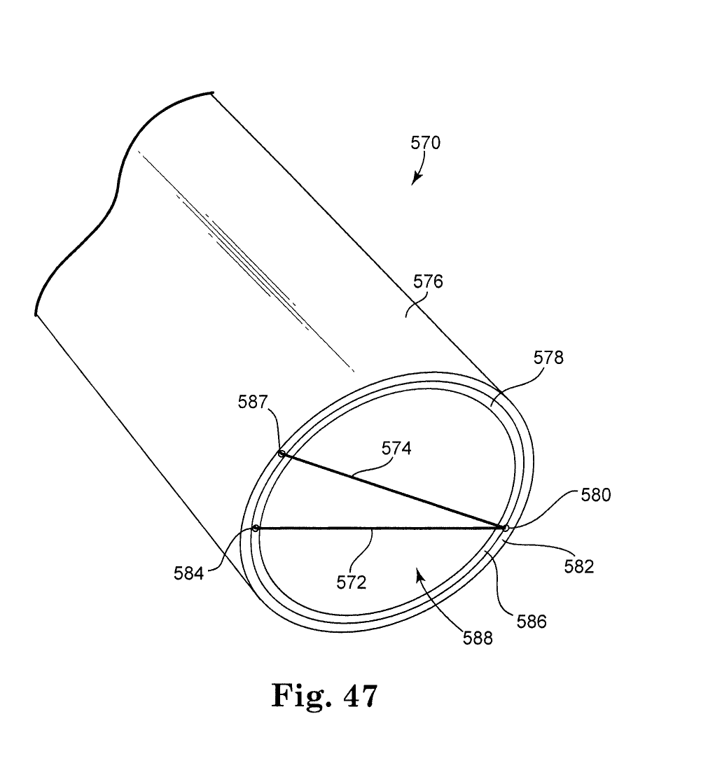

FIG. 47 illustrates another embodiment of a removal instrument 570 that utilizes a first cutting wire 572, a second cutting wire 574, an outer tubular member 576 having a perimeter 582, an inner tubular member 578 having a perimeter 586. The perimeters 582, 586 define a distal opening 588 through which an extension may be placed. One end of each of the first and second cutting wires 572, 574 are attached to the perimeter 582 of the outer tubular member 576 at a first fixing point 580. The opposing end of the first cutting wire 572 is attached to the inner tubular member 578 at a second fixing point 584, while the opposing end of the second cutting wire 574 is attached to the outer tubular member 576 at a third fixing point 587. The removal instrument 570 functions by rotating outer tubular member 576 and/or the inner tubular member 578 such that the second fixing point 584 moves past the third fixing point 587. In this way both the first and second cutting wires 572, 574 traverse the distal opening 588 of the removal instrument 570.

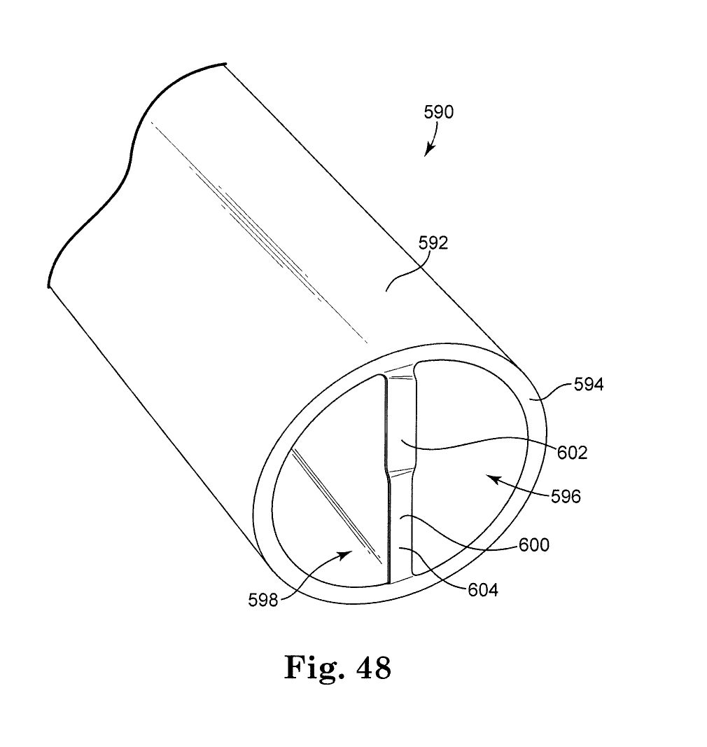

FIG. 48 illustrates another embodiment of a removal instrument 590 that includes a tubular member 592 having a perimeter 594 at a distal end of the tubular member 592. The removal instrument 590 also includes a transverse blade 600 that is attached to the perimeter 594. In the illustrated embodiment, the blade 600 may include a first portion 602 pitched in one direction and a second portion 604 pitched in another direction. In another embodiment, the blade 600 may be pitched in only one direction. In still another embodiment, the blade 600 may not be pitched. The perimeter 594 and the blade 600 define a first distal opening 596 and a second distal opening 598.

This embodiment of the removal instrument 590 functions by threading an extension and implant through the tubular member 592 and then through either the first distal opening 596 or the second distal opening 598. Once the implant is in the appropriate location, the removal instrument 590 may then be rotated to cut the extension. Alternatively, the removal instrument 590 can be inserted over the extension in order to initiate cutting.

Patents and patent applications disclosed herein, including those cited in the Background of the Invention, are hereby incorporated by reference. It is to be understood that the above description is intended to be illustrative, and not restrictive. Other embodiments of the invention are possible. Many other embodiments will be apparent to those of skill in the art upon reviewing the above description. The scope of the invention should, therefore, be determined with reference to the appended claims, along with the full scope of equivalents to which such claims are entitled.

What is claimed is:

1. An instrument for removing an extension coupled to a prosthesis, the instrument comprising:
at least one axial member having a proximal end, a distal end, a distal opening at the distal end adapted to receive the extension; and
at least one cutter positioned to traverse at least a portion of the distal opening when a force is applied near the proximal end of the axial member.

2. The instrument of claim 1, wherein the extension comprises a delivery catheter.

3. The instrument of claim 1, wherein the extension comprises biomaterial.

4. The instrument of claim 1, wherein the prosthesis is located in an intervertebral disc space.

5. The instrument of claim 1, wherein the force comprises torque.

6. The instrument of claim 1 wherein the axial member comprises a tubular member.

7. The instrument of claim 6, wherein the tubular member comprises an inner diameter adapted to receive the extension.

8. The instrument of claim 1 wherein the axial member comprises a rod member and an extension engaging member at the distal end of the rod member.

9. The instrument of claim 1 wherein the distal opening is less than about 10 millimeters.

10. The instrument of claim 1 wherein the cutter comprises at least one cutting wire having a first end and a second end and wherein at least the second end is accessible at the proximal end of the axial member, the cutting wire positioned to traverse at least a portion of the distal opening when a tension force is applied to the second end of the cutting wire.

11. The instrument of claim 10, wherein the first end of the cutting wire is accessible at the proximal end of the axial member.

12. The instrument of claim 10, wherein the axial member includes at least one channel and at least one of the first end and second end extend through the channel to the proximal end of the axial member.

13. The instrument of claim 10, wherein the axial member includes a first channel and a second channel and wherein the first end extends through the first channel and the second end extends through the second channel to the proximal end of the axial member.

14. The instrument of claim 1, wherein the cutter comprises at least one blade coupled to the at least one axial member that is positioned to traverse at least a portion of the distal opening when the force is applied.

15. The instrument of claim 14, wherein the at least one axial member comprises an outer tubular member and an inner axial member inside of the outer tubular member, wherein the outer tubular member comprises an interior surface that tapers toward a central axis of the outer tubular member and wherein the at least one blade is coupled to the inner axial member.

16. The instrument of claim 15, wherein the inner axial member comprises more than one blade.

17. The instrument of claim 15, wherein the at least one blade is parabolic.

18. The instrument of claim 15, wherein the at least one blade is triangular.

19. The instrument of claim 15, wherein the at least one blade is flat.

20. The instrument of claim 14, wherein the at least one axial member comprises an outer tubular member and an inner axial member inside of the outer tubular member, wherein the at least one blade is coupled to the outer tubular member.

21. The instrument of claim 14, wherein the at least one axial member comprises an outer tubular member and an inner axial member inside of the outer tubular member, wherein the at least one blade is coupled to the inner axial member.

22. The instrument of claim 14, wherein the at least one blade is coupled to the distal end of the at least one axial member.

23. The instrument of claim 22, wherein the instrument further comprises at least one removable blade guard covering the at least one blade.

24. The instrument of claim 1, wherein the at least one cutter is heated.

25. The instrument of claim 1, wherein the at least one cutter is mechanically agitated.

26. The instrument of claim 25, wherein the mechanical agitation comprises vibration, oscillation, or application of ultrasonic waves.

27. The instrument of claim 1, wherein the axial member further comprises a perimeter and wherein the cutter is positioned to traverse a portion of the distal opening that is proximal to the perimeter.

28. The instrument of claim 1, wherein the axial member further comprises a perimeter and wherein the cutter is positioned to traverse a portion of the distal opening that is angled with respect to the perimeter.

29. An instrument for cutting an extension coupled to a prosthesis, the instrument comprising:
an inner axial member having a proximal end and a distal end;
an outer tubular member having an inside surface adapted to receive the inner axial member, a proximal end, and a distal end; and
a cutter positioned at the distal end of the inner axial member or the outer tubular member and adapted to engage with the extension when a force is applied near the proximal end of the inner axial member or the outer tubular member.

30. The instrument of claim 29, wherein the inner axial member further comprises a distal opening at the distal end, a cutter adjacent to the distal opening, and an inside diameter adapted to receive the extension, such that when the force is applied near the proximal end of the outer tubular member, the cutter engages with the extension.

31. The instrument of claim 29, wherein the outer tubular member further comprises a perimeter having an inner edge that is capable of receiving the extension through a distal opening at the distal end and wherein the cutter is positioned on the inner edge such that when the force is applied near the proximal end of the inner axial member, the cutter engages with the extension.

32. The instrument of claim 29, wherein the outer tubular member further comprises a perimeter having an inner edge that is capable of receiving the extension through a distal opening at the distal end and wherein the cutter is positioned on the distal end of the inner axial member such that when the force is applied near the proximal end of the inner axial member, the cutter engages with the extension.

33. A method of removing an extension coupled to a prosthesis, the method comprising the steps of:
positioning at least one inner axial member having a proximal end, and a distal end adapted to receive the extension, around part of the extension so that the distal opening of the axial member is near the prosthesis; and
applying a force near the proximal end of the axial member so that at least one cutter traverses at least a portion of the distal opening such that at least a portion of the extension is separated from the prosthesis.

34. The method of claim 33, wherein the at least one cutter comprises a cutting wire.

35. The method of claim 33, further comprising heating the cutter.

36. The method of claim 33, further comprising mechanically agitating the cutter.

37. The method of claim 36, wherein the step of mechanically agitating includes vibrating, oscillating, or applying ultrasonic waves to the cutter.

38. The method of claim 33, wherein the at least one cutter comprises at least one blade.

39. The method of claim 33, further comprising the step of rotating the axial member around the extension.

40. The method of claim 33, wherein the method of removing an extension is performed using a minimally invasive technique.

41. The method of claim 33, wherein the force comprises torque.

42. The method of claim 33, wherein the force is applied using a mechanized device.
